SEQUENCE LISTING (25 sequences)

SEQ ID NO: 1 - protein, 1367 aa, synthetic construct
  REGION 1..1367: note = dCas9
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - DNA, 442 bp, synthetic construct
  misc_feature 1..442: note = sgRNA-8 PP7
  misc_difference 1..20: note = sgRNA targeting sequence
  source 1..442: mol_type = other DNA
nnnnnnnnnnnnnnnnnnnngagagctactgccatgaggagcagacgatatggcgtcgctccgatccgaccagcagagcatatgggctcgctggggctgcagcagcagaggatatggcctcgctgcggagtgacgagcagaccatatggggtcgctcggccaccagaagcagaagatatggcttcgcttccggacaactagcagatcatatgggatcgctaggagtagagtagcagatgatatggcatcgctacgtgtgacaagcagaacatatgggttcgcttgtcacacaaactactcaaatgtccgaaaggtggcaaacactccaaagcagccaaacggatcaaacatggcagtagcaagttcaaataaggctagtccgttatcaacttgaaaaagtggcaccgagtcggtgctttttttatcgatggcaagggaattc

SEQ ID NO: 3 - DNA, 155 bp, synthetic construct
  misc_feature 1..155: note = sgRNA-2 PP7
  misc_difference 1..20: note = sgRNA targeting sequence
  source 1..155: mol_type = other DNA
nnnnnnnnnnnnnnnnnnnngtttgagagctaccggagcagacgatatggcgtcgctccggtagcaagttcaaataaggctagtccgttatcaacttggagcagacgatatggcgtcgctccaagtggcaccgagtcggtgctttttttgaattc

SEQ ID NO: 4 - DNA, 399 bp, synthetic construct
  misc_feature 1..399: note = sgRNA-8 MS2
  misc_difference 1..20: note = sgRNA targeting sequence
  source 1..399: mol_type = other DNA
nnnnnnnnnnnnnnnnnnnngtttgagagctactgccatgaggaatgaccaccaggcattccgatccgacgatggaccatcaggccatcgagctgcagaagtgacgaccacgcacttcggagtgacagaggaggatcacccctctggccaccagagtagagcatcagcctactccggacaactacggaggaccaccccgtaggagtagagcgaggagcaccagccctcgcgtgtgacgatgacgatcacgcatcgtcacacaaactactcaaatgtccgaaaggtggcaaacactccaaagcagctaaacggatcaaacatggcagtagcaagttcaaataaggctagtccgttatcaacttgaaaaagtggcaccgagtcggtgctttttttgaattc

SEQ ID NO: 5 - DNA, 169 bp, synthetic construct
  misc_feature 1..169: note = sgRNA-2 MS2
  misc_difference 1..20: note = sgRNA targeting sequence
  source 1..169: mol_type = other DNA
nnnnnnnnnnnnnnnnnnnngtttgagagctaggccaacatgaggatcacccatgtctgcagggcctagcaagttcaaataaggctagtccgttatcaacttggccaacatgaggatcacccatgtctgcagggccaagtggcaccgagtcggtgctttttttgaattc

SEQ ID NO: 6 - DNA, 360 bp, synthetic construct
  misc_feature 1..360: note = sgRNA-8 BoxB
  misc_difference 1..20: note = sgRNA targeting sequence
  source 1..360: mol_type = other DNA
nnnnnnnnnnnnnnnnnnnngtttgagagctactgccatgagggccctgaaaaagggcccgatccgagggccctgaagaagggcccagctgcagggccctgaaaaagggcccggagtgagggccctgaagaagggcccgccaccagggccctgaaaaagggccccggacaagggccctgaagaagggcccgagtaggggccctgaaaaagggcccgtgtgagggccctgaagaagggcccacacaaactactcaaatgtccgaaaggtggcaaacactccaaagcagctaaacggatcaaacatggcagtagcaagttcaaataaggctagtccgttatcaacttgaaaaagtggcaccg

SEQ ID NO: 7 - DNA, 139 bp, synthetic construct
  misc_feature 1..139: note = sgRNA-2 BoxB
  misc_difference 1..20: note = sgRNA targeting sequence
  source 1..139: mol_type = other DNA
nnnnnnnnnnnnnnnnnnnngtttgagagctagggccctgaagaagggccctagcaagttcaaataaggctagtccgttatcaacttgggccctgaagaagggcccaagtggcaccgagtcggtgctttttttgaattc

SEQ ID NO: 8 - DNA, 314 bp, synthetic construct
  misc_feature 1..314: note = mU6
  source 1..314: mol_type = other DNA
gatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtt

SEQ ID NO: 9 - DNA, 241 bp, synthetic construct
  misc_feature 1..241: note = hU6
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 10 - protein, 28 aa, synthetic construct
  REGION 1..28: note = Foldon
GYIPEAPRDGQAYVRKDGEWVLLSTFLS

SEQ ID NO: 11 - protein, 32 aa, synthetic construct
  REGION 1..32: note = GCN4
MKQIEDKIEEILSKIYHIENEIARIKKLIGEV

SEQ ID NO: 12 - protein, 32 aa, synthetic construct
  REGION 1..32: note = 3HB
GEIAAIKQEIAAIKKEIAAIKWEIAAIKQGYG

SEQ ID NO: 13 - protein, 33 aa, synthetic construct
  REGION 1..33: note = 6G6H
TQEYLLKELMKLLKEQIKLLKEQIKMLKELEKQ

SEQ ID NO: 14 - protein, 135 aa, synthetic construct
  REGION 1..135: note = PCP
MGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGGGGTSGGGSGS

SEQ ID NO: 15 - protein, 126 aa, synthetic construct
  REGION 1..126: note = MCP
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGS

SEQ ID NO: 16 - protein, 31 aa, synthetic construct
  REGION 1..31: note = N22
MGNARTRRRERRAEKQAQWKAANGGGGTSGS

SEQ ID NO: 17 - DNA, 241 bp, synthetic construct
  misc_feature 1..241: note = sgRNA-3 PP7
  misc_difference 1..20: note = sgRNA targeting sequence
  source 1..241: mol_type = other DNA
nnnnnnnnnnnnnnnnnnnngtttgagagctactgccatgaggagcagacgatatggcgtcgctccgatccgaccagcagagcatatgggctcgctgggtgtgacaagcagaacatatgggttcgcttgtcacacaaacggatcaaacatggcagtagcaagttcaaataaggctagtccgttatcaacttgaaaaagtggcaccgagtcggtgctttttttatcgatggcaagggaattc

SEQ ID NO: 18 - DNA, 282 bp, synthetic construct
  misc_feature 1..282: note = sgRNA-4 PP7
  misc_difference 1..20: note = sgRNA targeting sequence
  source 1..282: mol_type = other DNA
nnnnnnnnnnnnnnnnnnnngtttgagagctactgccatgaggagcagacgatatggcgtcgctccgatccgaccagcagagcatatgggctcgctggggctgcagcagcagaggatatggcctcgctgcgtgtgacaagcagaacatatgggttcgcttgtcacacaaagcagccaaacggatcaaacatggcagtagcaagttcaaataaggctagtccgttatcaacttgaaaaagtggcaccgagtcggtgctttttttatcgatggcaagggaattc

SEQ ID NO: 19 - DNA, 323 bp, synthetic construct
  misc_feature 1..323: note = sgRNA-5 PP7
  misc_difference 1..20: note = sgRNA targeting sequence
  source 1..323: mol_type = other DNA
nnnnnnnnnnnnnnnnnnnngtttgagagctactgccatgaggagcagacgatatggcgtcgctccgatccgaccagcagagcatatgggctcgctggggctgcagcagcagaggatatggcctcgctgcggagtgacgagcagaccatatggggtcgctcggtgtgacaagcagaacatatgggttcgcttgtcacacaaacactccaaagcagccaaacggatcaaacatggcagtagcaagttcaaataaggctagtccgttatcaacttgaaaaagtggcaccgagtcggtgctttttttatcgatggcaagggaattc

SEQ ID NO: 20 - DNA, 364 bp, synthetic construct
  misc_feature 1..364: note = sgRNA-6 PP7
  misc_difference 1..20: note = sgRNA targeting sequence
  source 1..364: mol_type = other DNA
nnnnnnnnnnnnnnnnnnnngtttgagagctactgccatgaggagcagacgatatggcgtcgctccgatccgaccagcagagcatatgggctcgctggggctgcagcagcagaggatatggcctcgctgcggagtgacgagcagaccatatggggtcgctcggccaccagaagcagaagatatggcttcgcttcgtgtgacaagcagaacatatgggttcgcttgtcacacaaaggtggcaaacactccaaagcagccaaacggatcaaacatggcagtagcaagttcaaataaggctagtccgttatcaacttgaaaaagtggcaccgagtcggtgctttttttatcgatggcaagggaattc

SEQ ID NO: 21 - DNA, 405 bp, synthetic construct
  misc_feature 1..405: note = sgRNA-7 PP7
  misc_difference 1..20: note = sgRNA targeting sequence
  source 1..405: mol_type = other DNA
nnnnnnnnnnnnnnnnnnnngtttgagagctactgccatgaggagcagacgatatggcgtcgctccgatccgaccagcagagcatatgggctcgctggggctgcagcagcagaggatatggcctcgctgcggagtgacgagcagaccatatggggtcgctcggccaccagaagcagaagatatggcttcgcttccggacaactagcagatcatatgggatcgctaggtgtgacaagcagaacatatgggttcgcttgtcacacaaatgtccgaaaggtggcaaacactccaaagcagccaaacggatcaaacatggcagtagcaagttcaaataaggctagtccgttatcaacttgaaaaagtggcaccgagtcggtgctttttttatcgatggcaagggaattc

SEQ ID NO: 22 - protein, 416 aa, synthetic construct
  REGION 1..416: note = Foldon-GFP-PCP
MGYIPEAPRDGQAYVRKDGEWVLLSTFLSGGGGSGGGGSGGGGSRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKMGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGGGGTSGGGSGS

SEQ ID NO: 23 - protein, 448 aa, synthetic construct
  REGION 1..448: note = PCP-foldon-GFP
MGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGGGGTSGGGSGSSGGSSGGSSGSETPGTSESATPESSGGSSGGSMGYIPEAPRDGQAYVRKDGEWVLLSTFLSGGGGSGGGGSGGGGSRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 24 - protein, 54 aa, synthetic construct
SPKIKPFHFTKDVQSGEREQVTCSVKLGDPPFTYTWKKDGIDINEFKDIKIEIS

SEQ ID NO: 25 - protein, 442 aa, synthetic construct
SPKIKPFHFTKDVQSGEREQVTCSVKLGDPPFTYTWKKDGIDINEFKDIKIEISSGGGGSGGGGSGGGGSRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKMGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGGGGTSGGGSGS

(19) United States
(12) Patent Application Publication
(10) Pub. No.: US 2025/0327117 A1
(43) Pub. Date: Oct. 23, 2025

(54) CRISPR-BASED IMAGING SYSTEM AND USE THEREOF

(71) Applicant: Westlake Laboratory of Life Sciences and Biomedicine, Hangzhou, Zhejiang (CN)

(72) Inventors: Chunqing Song, Hangzhou, Zhejiang (CN); Enzhi Shen, Hangzhou, Zhejiang (CN); Xinyuan Lyu, Hangzhou, Zhejiang (CN); Yuan Deng, Hangzhou, Zhejiang (CN)

(21) Appl. No.: 18/857,129

(22) PCT Filed: Apr. 17, 2023
(86) PCT No.: PCT/CN2023/088712

§ 371 Date: Oct. 15, 2024

(30) Foreign Application Priority Data
Apr. 15, 2022 (CN) 202210413917.9

(51) Int. Cl. C12Q 1/6841; C12Q 1/6825

(52) U.S. Cl. CPC C12Q 1/6841; C12Q 1/6825

(57) ABSTRACT

Provided are a CRISPR-based imaging system and use thereof. The imaging system comprises: (1) a dCas9-expressing vector or a dCas9 protein; (2) an engineered sgRNA-expressing vector, the engineered sgRNA comprising: a sgRNA backbone containing n copies of RNA aptamer, and a sgRNA sequence specific for a target gene to be detected, wherein n is an integer greater than or equal to 2; and (3) a fusion protein-expressing vector, the fusion protein comprising: an RNA-binding motif specifically recognizing the RNA aptamer, a multimerization peptide and a fluorescent protein, which are operably linked to each other. The imaging system has improved resolution, and achieves labeling and imaging of non-repetitive sequence, especially labeling and imaging of non-repetitive sequence within single-copy gene loci in living cells.

### SEQUENCE LISTING

This application contains a Sequence Listing that has been submitted electronically as an XML file named “59084-0002US1.XML.” The XML file, created on Jul. 3, 2025, is 42,425 bytes in size. The material in the XML file is hereby incorporated by reference in its entirety.

### TECHNICAL FIELD

The present invention relates to a CRISPR-based imaging system and use thereof. Specifically, the CRISPR-based imaging system of the present invention is a CRISPR-based fluorescence in situ hybridization amplifier system, briefly referred to as the CRISPR FISHer system.

### BACKGROUND ART

Since the successful implementation of the Human Genome Project, great progress has been made in the field of life sciences, especially in the field of molecular biology. People have a deeper understanding of the processes of gene replication, repair, transcription, and translation. The study of these important biological processes is inseparable from the development and application of DNA or RNA sequence-specific or structure-specific imaging technologies. At present, people have developed a variety of imaging techniques (e.g., fluorescent in situ hybridization, etc., which can realize the DNA imaging in fixed cells and the location imaging of repetitive sequences-containing genomes in living cells). Still, most gene sequences (about 65%) are non-repetitive sequences [1], their imaging in living cells is of great significance for understanding the behavior of genes in chromatin and how they participate in transcriptional regulation, etc., Still, due to technical limitations, the non-repetitive region live cell imaging is difficult to be realized.

### I. Traditional Imaging Technique-Fluorescence In Situ Hybridization (FISH)-Labeling Endogenous Genomic Loci

Nowadays, fluorescence in situ hybridization (FISH) technology has been widely used in biological gene labeling [2,3]. This method uses fluorescently labeled specific nucleic acid probes to hybridize with corresponding target DNA molecules in cells, so as to determine the intracellular localization of the DNA region bound by the fluorescent probe. However, since the signal of a single fluorescent molecule is very weak, in order to obtain higher resolution, scientists often design multiple fluorescent probes and make them simultaneously target multiple adjacent sequences in the target site [4]. Although FISH has been widely used in gene labeling, many problems remain. For example: 1) This method needs to fix the cells for observation, so it can only obtain the qualitative target DNA state of the cells at a certain moment; 2) After the cells are fixed, the DNA undergoes denaturation, and the structural state of the chromatin is challenging to remain intact.

Ii. CRISPR/Cas-Based Live Cell Imaging Technology

With the promotion of CRISPR/Cas gene editing technology, scientists have discovered that the nuclease-inactivated form of Cas9 (Dead Cas9, referred to as dCas9) can still bind to single guide RNA (referred to as sgRNA) and specifically bind to the genome sequence complementary to sgRNA [5], and then promote the imaging technology of genomic loci in live cells.

### (1) Fluorescent Protein-Based CRISPR Imaging System

In 2013, Chen Baohui et al. [6] first performed the fused expression of dCas9 and EGFP, and with the help of the guiding of sgRNA that targets telomere repeat sequence, the genome imaging of telomere could be observed. Chen Baohui et al. first applied the CRISPR system to the imaging field to label telomeres with more repetitive sequences, and realized gene imaging in living cells for the first time [6]. However, the resolution of this system can only label sites with repetitive sequences like telomeres, and the presence of free fluorescently labeled dCas9, EGFP or dCas9-EGFP complexes not bound to target inevitably increases the background signal. The dCas9 protein tends to localize in the nucleolus, and a series of studies have observed high background signals induced by dCas9-EGFP in the nucleolus [6,7]. Many scientists have tried to use the dCas9-sun-tag system (based on the interaction of GCN4 and scFv) to recruit more fluorescent proteins bound to dCas9 [8,9], but the background signal of this system is very high.

In addition to using dCas9 to fuse fluorescent proteins, many research groups modify sgRNA by adding a binding functional region that RNA-binding proteins can recognize, and the modified sgRNA can recruit fusion proteins of fluorescent proteins and RNA-binding proteins to the genomic target sequence to realize the labeling at different sites in the genome [10-12]. Among them, the most widely used sgRNA modification is the addition of MS2 ligand, which is an RNA stem-loop structure derived from the bacteriophage MS2 RNA virus, and which can bind to the MS2 coat protein (MCP) with high specificity and affinity [13].

In 2018, Ma Hanhui et al. developed the CRISPR-Sirius imaging system, which maintains the advantages of multi-color and flexibility and increases the resolution limit of the CRISPR imaging system to 22 copies. However, it remains the most critical issue in DNA imaging in living cells to improve the signal/background ratio and achieve the single-copy resolution.

### (2) Organic Dye-Based CRISPR-dCas9 System

Organic dyes are generally brighter, more photostable, and smaller in size than fluorescent proteins. Currently, three dye-based organic systems have demonstrated the feasibility of visualizing genomic loci in living cells. They include Halo tag-based system, RNA ligand-based system and molecular beacon-based system. First, in the Halo tag system, dCas9 can be fused with a Halo tag, the Halo tag is a mutant of bacterial haloalkane dehalogenase, which can be covalently bound to a Halo tag ligand, the Halo tag ligand is a cell-permeable chloroalkane molecule that can be chemically attached to the dye of choice [14]. Second, the RNA ligand-based system uses a dye based on 3,5-difluoro-4-hydroxybenzylimidazolidinone (DFHBI), which is a reactive dye that can be quenched under physiological conditions, but will fluoresce when binding to a homologous RNA nucleic acid ligand [15]. Its labeling principle is similar to that of the Halo tag system. However, the two systems have low relative signal/background values and thus cannot be used for higher resolution labeling.

In order to further improve the signal/background ratio, scientists developed the MBs CRISPR/dCas9 system. MBs are a class of quenchable fluorescent oligonucleotide probes, which can activate fluorescence after binding to complementary nucleic acid targets [16]. Still, they can hardly achieve the specific fluorescent labeling of non-repetitive sequences of genomes.

### (3) Nanoparticle-Based CRISPR-dCas9 System

Quantum dot (QD) is a kind of luminescent semiconductor nanoparticle with a size of 50-100 nm, which has brightness and photostability superior to synthetic dyes and fluorescent proteins. However, as a class of synthetic nanomaterials, QDs also have similar limitations as the synthetic dyes, for example, quantum dots may hardly be delivered effectively due to their large size [17].

Iii. Current Problems in Imaging Technology Based on the CRISPR-Cas9 System

Although great progress has been made in the field of live cell imaging based on the CRISPR-Cas9 system, many challenges remain to be overcome.

### (1) Low Signal/Background Ratio, Low Resolution (Presence of Strong Background Signal)

To improve the signal-to-background ratio, scientists have been working on increasing the signal through fluorescent labeling of dCas9 or sgRNA. This strategy inevitably increases the background signal due to the presence of free fluorescently labeled dCas9, sgRNA, or dCas9-sgRNA complexes not bound to the target. It has been speculated that reducing background signals may require more sophisticated imaging methods such as fluorescence resonance energy transfer (FRET), which has been used for background-free imaging of RNA and proteins [18,19].

### (2) Existing Challenges on Imaging of Non-Repetitive Sequences

Compared with repetitive sequences that can be imaged with only one sgRNA, non-repetitive sequences may require multiple different sgRNAs to target at the same time, which is very difficult to achieve. Current research includes cloning multiple sgRNAs into gRNA oligos (CARGO) to simplify the transfection process and improve the transfection efficiency. Despite these advances, the simultaneous expression of multiple different sgRNA species in a single cell remains challenging because the transcription rate of RNA often exhibits jumpy variations [20,21]. Therefore, the production of multiple sgRNAs may be “out of sync” between each other. To increase the co-expression of different sgRNAs, one possible strategy is to construct an expression vector in one transcript, in which every two sgRNAs are linked by a matrix, and the matrix can be excised by RNases. tRNA is one of the candidates for this substrate [22]. Even if all different sgRNAs can be expressed simultaneously, imaging of non-repetitive sequences is still challenging because different sgRNAs may compete with each other for binding to dCas9, thereby still failing to achieve signal amplification.

Therefore, there is a need for a system and method capable of improving the resolution of imaging systems, especially achieving non-repetitive locus labeling and imaging.

### DESCRIPTION OF INVENTION

The object of the present invention is to improve the resolution of imaging systems and achieve the labeling and imaging of non-repetitive region of single-copy gene.

In one aspect, the present invention provides a CRISPR-based imaging system (full name is CRISPR based fluorescent in situ hybridization amplifier system, briefly referred to as CRISPR FISHer system), the imaging system is capable of improving the resolution of imaging systems, achieve the labeling and imaging of single-copy non-repetitive gene loci, especially in a living cell.

The CRISPR-based imaging system of the present invention comprises:
(1) a dCas9-expressing vector or a dCas9 protein;
(2) an engineered sgRNA-expressing vector, the engineered sgRNA comprising: a sgRNA backbone containing n copies of RNA aptamer and a sgRNA sequence specific for a target gene to be detected, wherein n is an integer greater than or equal to 2;
(3) a fusion protein-expressing vector, the fusion protein comprising: an RNA binding motif that specifically recognizes the RNA aptamer, a multimerization peptide segment, and a fluorescent protein, which are operably linked between each other in a manner that is not limited, and an optimal linking manner may be selected according to practical needs.

In one embodiment, in the CRISPR FISHer system, the dCas9-expressing vector or dCas9 protein can be replaced with a cell line stably expressing the dCas9 protein. The dCas9 is set forth in SEQ ID No: 1.

The engineered sgRNA described in the present invention does not change the sequence binding to dCas9, a stem-loop part of the sgRNA is modified by inserting an RNA aptamer sequence therein.

In one embodiment, the engineered sgRNA-expressing vector is driven by a U6 promoter, which may be a mouse U6 promoter (mU6) or a human U6 promoter (hU6);
the RNA aptamer is located in the sgRNA backbone stem-loop (i.e., sgRNA scaffold), and the RNA aptamer can be selected from, but not limited to: PP7, MS2 or BoxB;
n copies of RNA aptamer mean that n copies of RNA aptamer are linked in series, which can be linked through linkers or linked directly. When linked through a linker, the linker can be selected from linkers commonly used in the art. Wherein, n is an integer greater than or equal to 2, for example, it can be 2, 3, 4, 5, 6, 7 or 8 or greater integer, its upper limit is not particularly limited, and those skilled in the art can choose a suitable value of n according to practical needs;
the multimerization peptide segment can be selected from, but not limited to, foldon trimerization small peptide, GCN4 trimerization small peptide, 3HB trimerization small peptide, 6G6H hexamerization small peptide, or sDscama30 dimerization small peptide, wherein foldon consists of an amino acid sequence as set forth in SEQ ID No: 10, and GCN4, 3HB, 6G6H and sDscama30 are set forth in SEQ ID No: 11, 12, 13 and 24, respectively;
wherein the RNA binding motif in the fusion protein specifically recognizes an RNA aptamer in the engineered sgRNA, that is, the RNA aptamer and the RNA binding motif are paired, so that the RNA binding motif in the fusion protein can be, but not limited to: PCP that recognizes PP7, MCP that recognizes MS2, or N22 that recognizes BoxB; wherein the amino acid sequences of PCP, MCP and N22 are set forth in SEQ ID No: 14, 15, and 16, respectively; in other words, in the CRISPR-based labeling and imaging system of the present invention, the RNA aptamer and the RNA binding motif exist in a paired combination, and the combination is selected from: PP7 and PCP, MS2 and MCP, or BoxB and N22.

The fluorescent protein in the fusion protein can be selected from, but not limited to: green fluorescent protein (GFP), enhanced green fluorescent protein (EGFP), red fluorescent protein (RFP), blue fluorescent protein (BFP), etc.

According to the needs of practical applications, those skilled in the art can easily select appropriate plasmids to construct the expression vectors of (1) to (3). Available plasmids include, but are not limited to, pX330, pUR, and lentivirus lenti, etc.,

In one embodiment, in the fusion protein-expressing vector, the multimerization peptide segment can be fused to the N-terminal or C-terminal of the fluorescent protein, or located at the N-terminal or C-terminal of the fusion protein, preferably, the multimerization peptide is located at the N-terminus of the fusion protein. For example, from the N-terminal to the C-terminal, the structure of the fusion protein can be: RNA binding motif-multimerization peptide segment-fluorescent protein, RNA binding motif-fluorescent protein-multimerization peptide segment, multimerization peptide segment-RNA binding motif-fluorescent protein, or multimerization peptide segment-fluorescent protein-RNA binding motif.

In one embodiment, the fusion protein-expressing vector further comprises a nuclear localization sequence (NLS), and the nuclear localization sequence (NLS) can be located at the N-terminal or C-terminal of the fusion protein.

In one embodiment, the CRISPR-based imaging system of the present invention comprises:
(1) a dCas9-expressing vector or a dCas9 protein,
(2) an engineered sgRNA-expressing vector, the engineered sgRNA has a structure shown in U6-sgRNA-n×PP7, wherein U6 is a promoter, sgRNA is a guide RNA specific for a target gene to be detected, and the sgRNA is capable of binding to dCas9, PP7 is an RNA aptamer, n×PP7 represents that n copies of PP7 are inserted in series in the sgRNA backbone stem-loop, wherein n is an integer greater than or equal to 2, and
(3) a fusion protein-expressing vector, in which the fusion protein from N-terminus to C-terminus is foldon-fluorescent protein-PCP or PCP-foldon-fluorescent protein.

Wherein, n can be 2, 3, 4, 5, 6, 7 or 8 or greater integer, and its upper limit is not particularly limited, and those skilled in the art can select a suitable value of n according to practical needs; the fluorescent protein can be selected according to practical needs, for example, green fluorescent protein (GFP), enhanced green fluorescent protein (EGFP), red fluorescent protein (RFP) or blue fluorescent protein (BFP), etc.

In a specific embodiment, n is 2 or 8.

In a specific embodiment, the CRISPR-based imaging system of the present invention comprises:
(1) a dCas9-expressing vector or a dCas9 protein,
(2) an engineered sgRNA-expressing vector, in which the engineered sgRNA has a structure shown in U6-sgRNA-2×PP7, wherein U6 is a promoter, sgRNA is a guide RNA specific for a target gene to be detected, and the sgRNA is capable of binding to dCas9, PP7 is an RNA aptamer, 2×PP7 represents that 2 copies of PP7 are inserted in series in the sgRNA backbone stem-loop, and
(3) a fusion protein-expressing vector, in which the fusion protein from N-terminus to C-terminus is foldon-fluorescent protein-PCP.

In a specific embodiment, the CRISPR-based imaging system of the present invention comprises:
(1) a dCas9-expressing vector or a dCas9 protein,
(2) an engineered sgRNA-expressing vector, in which the engineered sgRNA has a structure shown in U6-sgRNA-8×PP7, wherein U6 is a promoter, sgRNA is a guide RNA specific for a target gene to be detected, and the sgRNA is capable of binding to dCas9, PP7 is an RNA aptamer, 8×PP7 represents that 8 copies of PP7 are inserted in series in the sgRNA backbone stem-loop, and
(3) a fusion protein-expressing vector, in which the fusion protein from N-terminus to C-terminus is foldon-fluorescent protein-PCP.

In a specific embodiment, the CRISPR-based imaging system of the present invention comprises:
(1) a dCas9-expressing vector or a dCas9 protein,
(2) an engineered sgRNA-expressing vector, in which the engineered sgRNA has a structure shown in U6-sgRNA-8×PP7, wherein U6 is a promoter, sgRNA is a guide RNA specific for a target gene to be detected, and the sgRNA is capable of binding to dCas9, PP7 is an RNA aptamer, 8×PP7 represents that 8 copies of PP7 are inserted in series in the sgRNA backbone stem-loop, and
(3) a fusion protein-expressing vector, in which the fusion protein from N-terminus to C-terminus is PCP-foldon-fluorescence protein.

In one embodiment, the CRISPR-based imaging system of the present invention comprises:
(1) a dCas9-expressing vector or a dCas9 protein,
(2) an engineered sgRNA-expressing vector, in which the engineered sgRNA has a structure shown in U6-sgRNA-nxMS2, wherein U6 is a promoter, sgRNA is a guide RNA specific for a target gene to be detected, and the sgRNA is capable of binding to dCas9, MS2 is an RNA aptamer, n×MS2 represents that n copies of MS2 are inserted in series in the sgRNA backbone stem-loop, wherein n is an integer greater than or equal to 2, and
(3) a fusion protein-expressing vector, in which the fusion protein from N-terminus to C-terminus is foldon-fluorescent protein-MCP or MCP-foldon-fluorescent protein.

Wherein, n can be 2, 3, 4, 5, 6, 7 or 8 or greater integer, and its upper limit is not particularly limited, and those skilled in the art can select a suitable value of n according to practical needs; the fluorescent protein can be selected according to practical needs, for example, green fluorescent protein (GFP), enhanced green fluorescent protein (EGFP), red fluorescent protein (RFP) or blue fluorescent protein (BFP), etc.

In a specific embodiment, n is 2 or 8.

In one embodiment, the CRISPR-based imaging system of the present invention comprises:
(1) a dCas9-expressing vector or a dCas9 protein,
(2) an engineered sgRNA-expressing vector, in which the engineered sgRNA has a structure shown in U6-sgRNA-nxBoxB, wherein U6 is a promoter, sgRNA is a guide RNA specific for a target gene to be detected, and the sgRNA is capable of binding to dCas9, BoxB is an RNA aptamer, nxBoxB represents that n copies of BoxB are inserted in series in the sgRNA backbone stem-loop, where n is an integer greater than or equal to 2, and
(3) a fusion protein-expressing vector, in which the fusion protein from N-terminus to C-terminus is foldon-fluorescent protein-N22 or N22-foldon-fluorescent protein.

Likewise, wherein, n can be 2, 3, 4, 5, 6, 7 or 8 or greater integer, its upper limit is not particularly limited, and those skilled in the art can select a suitable value of n according to practical needs; the fluorescent protein can be selected according to practical needs, for example, green fluorescent protein (GFP), enhanced green fluorescent protein (EGFP), red fluorescent protein (RFP) or blue fluorescent protein (BFP), etc.

In a specific embodiment, n is 2 or 8.

In another embodiment, the multimerization peptide segment foldon in the fusion protein-expressing vector can be replaced by GCN4, 3HB, 6G6H or sDscama30.

In another embodiment, in the fusion protein-expressing vector, the multimerization peptide segment foldon, GCN4, 3HB, 6G6H or sDscama30 can be fused to the N-terminal or C-terminal of the fluorescent protein, or located at the N-terminal or C-terminal of the entire fusion protein, preferably at the N-terminus of the entire fusion protein.

In another embodiment, the CRISPR-based imaging system of the present invention comprises:
(1) a dCas9-expressing vector or a dCas9 protein,
(2) an engineered sgRNA-expressing vector, the engineered sgRNA has a structure shown in U6-sgRNA-n×PP7, wherein U6 is a promoter, sgRNA is a guide RNA specific for a target gene to be detected, and the sgRNA is capable of binding to dCas9, PP7 is an RNA aptamer, n×PP7 represents that n copies of PP7 are inserted in series in the sgRNA backbone stem-loop, wherein n is an integer greater than or equal to 2, and
(3) a fusion protein-expressing vector, in which the fusion protein from N-terminus to C-terminus is sDscama30-fluorescent protein-PCP.

Wherein, n can be 2, 3, 4, 5, 6, 7 or 8 or greater integer, and its upper limit is not particularly limited, and those skilled in the art can select a suitable value of n according to practical needs; the fluorescent protein can be selected according to practical needs, for example, green fluorescent protein (GFP), enhanced green fluorescent protein (EGFP), red fluorescent protein (RFP) or blue fluorescent protein (BFP), etc.

Alternatively, PP7 and PCP in the above embodiment may be replaced with MS2 and MCP, respectively, or may be replaced with BoxB and N22, respectively.

In a specific embodiment, the CRISPR-based imaging system of the present invention comprises:
(1) a dCas9-expressing vector or a dCas9 protein,
(2) an engineered sgRNA-expressing vector, the engineered sgRNA has a structure shown in U6-sgRNA-7×PP7, wherein U6 is a promoter, sgRNA is a guide RNA specific for a target gene to be detected, and the sgRNA is capable of binding to dCas9, PP7 is an RNA aptamer, 7×PP7 represents that 7 copies of PP7 are inserted in series in the sgRNA backbone stem-loop, wherein n is an integer greater than or equal to 2, and
(3) a fusion protein-expressing vector, in which the fusion protein from N-terminus to C-terminus is sDscama30-fluorescent protein-PCP.

Those skilled in the art can understand that the plasmids used to construct the dCas9-expressing vector, the engineered sgRNA-expressing vector and the fusion protein-expressing vector are not particularly limited, and those skilled in the art can select appropriate plasmids to construct these expression vectors. For example, the plasmid used to construct the sgRNA-n×PP7-expressing vector can be found on the Addgene website, for example, the plasmid under No. #121943 can be used.

For the CRISPR-based imaging system of the present invention, it should be noted that:
1) The RNA aptamer in the engineered sgRNA-expressing vector is paired with the RNA binding motif in the fusion protein to realize the specific recognition of the RNA aptamer by the RNA binding motif. The combination of RNA aptamer and RNA binding motif that can be used is: PP7 and PCP, MS2 and MCP, or BoxB and N22. The combinations of other similar RNA aptamers and RNA binding motifs also can be used for the CRISPR-based labeling and imaging system of the present invention.
2) For the dCas protein element, CMV promoter, EFla promoter, etc. can be conventionally used to continuously promote the expression of dCas9 protein, or an inducible promoter can be used to promote the specific expression of dCas9. Those skilled in the art will be able to select an appropriate promoter.
3) The multimerization peptide segment is not limited to foldon trimerization small peptide, while GCN4 (trimerization), 3HB (trimerization), 6G6H (hexamerization), or sDscama30 (dimerization), etc. can also be used. These multimerization peptide segments can make a fusion peptide containing the multimerization peptide segments that exist in the form of a multimer.
4) The promoter of sgRNA can be mouse U6 promoter (mU6) or human U6 promoter (hU6).
5) There is no particular limitation on the plasmids used to construct the relevant expression vectors in the CRISPR-based imaging system of the present invention, and those skilled in the art can easily select appropriate plasmids according to the needs of practical applications.

The amino acid or nucleotide sequences of the relevant elements in the CRISPR-based imaging system of the present invention are as follows:

dCas9
(SEQ ID No: 1)
DKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNLS
DAILLSDILRVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNGSIPHQIHLG
ELHAILRRQEDFYPFLKDNREKIEKILTFRIPYYVGPLARGNSRFAWMTRKSEETITPWNF
EEVVDKGASAQSFIERMTNFDKNLPNEKVLPKHSLLYEYFTVYNELTKVKYVTEGMRK
PAFLSGEQKKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKG
SPEDNEQKQLFVEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLYETRIDLSQLGGD
sgRNA-8xPP7
(SEQ ID No: 2)
NNNNNNNNNNNNNNNNNNNNGAGAGCTACTGCCATGAGGAGCAGACGATATG
GCGTCGCTCCGATCCGACCAGCAGAGCATATGGGCTCGCTGGGGCTGCAGCAGCAG
AGGATATGGCCTCGCTGCGGAGTGACGAGCAGACCATATGGGGTCGCTCGGCCACC
AGAAGCAGAAGATATGGCTTCGCTTCCGGACAACTAGCAGATCATATGGGATCGCT
AGGAGTAGAGTAGCAGATGATATGGCATCGCTACGTGTGACAAGCAGAACATATGG
GTTCGCTTGTCACACAAACTACTCAAATGTCCGAAAGGTGGCAAACACTCCAAAGC
AGCCAAACGGATCAAACATGGCAGTAGCAAGTTCAAATAAGGCTAGTCCGTTATCA
ACTTGAAAAAGTGGCACCGAGTCGGTGCTTTTTTTATCGATGGCAAGGGAATTC
wherein, NNNNNNNNNNNNNNNNNNNN represents a sgRNA targeting sequence, the
same below. The underlined sequences are the stem-loop structure sequences of PP7,
showing 8 copies of PP7 are linked via a linker in series.
sgRNA-2xPP7
(SEQ ID No: 3)
NNNNNNNNNNNNNNNNNNNNGTTTGAGAGCTACCGGAGCAGACGATATGGCG
TCGCTCCGGTAGCAAGTTCAAATAAGGCTAGTCCGTTATCAACTTGGAGCAGACGAT
ATGGCGTCGCTCCAAGTGGCACCGAGTCGGTGCTTTTTTTGAATTC
wherein, the underlined sequences are the stem-loop structure sequences of PP7,
showing 2 copies of PP7 are linked via a linker in series.
sgRNA-8xMS2 (SEQ ID No: 4)
NNNNNNNNNNNNNNNNNNNNGTTTGAGAGCTACTGCCATGAGGAATGACCAC
CAGGCATTCCGATCCGACGATGGACCATCAGGCCATCGAGCTGCAGAAGTGACGAC
CACGCACTTCGGAGTGACAGAGGAGGATCACCCCTCTGGCCACCAGAGTAGAGCAT
CAGCCTACTCCGGACAACTACGGAGGACCACCCCGTAGGAGTAGAGCGAGGAGCAC
CAGCCCTCGCGTGTGACGATGACGATCACGCATCGTCACACAAACTACTCAAATGTC
CGAAAGGTGGCAAACACTCCAAAGCAGCTAAACGGATCAAACATGGCAGTAGCAA
GTTCAAATAAGGCTAGTCCGTTATCAACTTGAAAAAGTGGCACCGAGTCGGTGCTTT
TTTTGAATTC
wherein, the underlined sequences are the stem-loop structure sequences of MS2,
showing 8 copies of MS2 are linked via a linker in series.
sgRNA-2xMS2 (SEQ ID No: 5)
NNNNNNNNNNNNNNNNNNNNGTTTGAGAGCTAGGCCAACATGAGGATCACCC
ATGTCTGCAGGGCCTAGCAAGTTCAAATAAGGCTAGTCCGTTATCAACTTGGCCAAC
ATGAGGATCACCCATGTCTGCAGGGCCAAGTGGCACCGAGTCGGTGCTTTTTTTGAA
TTC
wherein, the underlined sequences are the stem-loop structure sequences of MS2,
showing 2 copies of MS2 are linked via a linker in series.
sgRNA-8xBoxB
(SEQ ID No: 6)
NNNNNNNNNNNNNNNNNNNNGTTTGAGAGCTACTGCCATGAGGGCCCTGAAA
AAGGGCCCGATCCGAGGGCCCTGAAGAAGGGCCCAGCTGCAGGGCCCTGAAAAAG
GGCCCGGAGTGAGGGCCCTGAAGAAGGGCCCGCCACCAGGGCCCTGAAAAAGGGC
CCCGGACAAGGGCCCTGAAGAAGGGCCCGAGTAGGGGCCCTGAAAAAGGGCCCGT
GTGAGGGCCCTGAAGAAGGGCCCACACAAACTACTCAAATGTCCGAAAGGTGGCAA
ACACTCCAAAGCAGCTAAACGGATCAAACATGGCAGTAGCAAGTTCAAATAAGGCT
AGTCCGTTATCAACTTGAAAAAGTGGCACCG
wherein, the underlined sequences are the stem-loop structure sequences of BoxB,
showing 8 copies of BoxB are linked via a linker in series.
sgRNA-2xBoxB
(SEQ ID No: 7)
NNNNNNNNNNNNNNNNNNNNGTTTGAGAGCTAGGGCCCTGAAGAAGGGCCCT
AGCAAGTTCAAATAAGGCTAGTCCGTTATCAACTTGGGCCCTGAAGAAGGGCCCAA
GTGGCACCGAGTCGGTGCTTTTTTTGAATTC
wherein, the underlined sequences the stem-loop structure sequences of BoxB,
showing 2 copies of BoxB are linked via a linker in series.
mU6 promoter
(SEQ ID No: 8)
GATCCGACGCCGCCATCTCTAGGCCCGCGCCGGCCCCCTCGCACAGACTTGTG
GGAGAAGCTCGGCTACTCCCCTGCCCCGGTTAATTTGCATATAATATTTCCTAGTAA
CTATAGAGGCTTAATGTGCGATAAAAGACAGATAATCTGTTCTTTTTAATACTAGCT
ACATTTTACATGATAGGCTTGGATTTCTATAAGAGATACAAATACTAAATTATTATT
TTAAAAAACAGCACAAAAGGAAACTCACCCTAACTGTAAAGTAATTGTGTGTTTTG
AGACTATAAATATCCCTTGGAGAAAAGCCTTGTT
hU6 promoter
(SEQ ID No: 9)
GAGGGCCTATTTCCCATGATTCCTTCATATTTGCATATACGATACAAGGCTGTT
AGAGAGATAATTGGAATTAATTTGACTGTAAACACAAAGATATTAGTACAAAATAC
GTGACGTAGAAAGTAATAATTTCTTGGGTAGTTTGCAGTTTTAAAATTATGTTTTAA
AATGGACTATCATATGCTTACCGTAACTTGAAAGTATTTCGATTTCTTGGCTTTATA
TATCTTGTGGAAAGGAC
Foldon
(SEQ ID No: 10)
GYIPEAPRDGQAYVRKDGEWVLLSTFLS
GCN4
(SEQ ID No: 11)
MKQIEDKIEEILSKIYHIENEIARIKKLIGEV
3HB
(SEQ ID No: 12)
GEIAAIKQEIAAIKKEIAAIKWEIAAIKQGYG
6G6H
(SEQ ID No: 13)
TQEYLLKELMKLLKEQIKLLKEQIKMLKELEKQ
PCP
(SEQ ID No: 14)
MGSKTIVLSVGEATRTLTEIQSTADRQIFEEKVGPLVGRLRLTASLRQNGAKTAYR
VNLKLDQADVVDSGLPKVRYTQVWSHDVTIVANSTEASRKSLYDLTKSLVATSQVED
LVVNLVPLGRGGGGTSGGGSGS
MCP
(SEQ ID No: 15)
MASNFTQFVLVDNGGTGDVTVAPSNFANGIAEWISSNSRSQAYKVTCSVRQSSAQ
NRKYTIKVEVPKGAWRSYLNMELTIPIFATNSDCELIVKAMQGLLKDGNPIPSAIAANS
GIYGGSGSGAGS
N22
(SEQ ID No: 16)
MGNARTRRRERRAEKQAQWKAANGGGGTSGS
sgRNA-3xPP7
(SEQ ID No: 17)
NNNNNNNNNNNNNNNNNNNNGTTTGAGAGCTACTGCCATGAGGAGCAGACG
ATATGGCGTCGCTCCGATCCGACCAGCAGAGCATATGGGCTCGCTGGGTGTGACAA
GCAGAACATATGGGTTCGCTTGTCACACAAACGGATCAAACATGGCAGTAGCAAG
TTCAAATAAGGCTAGTCCGTTATCAACTTGAAAAAGTGGCACCGAGTCGGTGCTTT
TTTTATCGATGGCAAGGGAATTC
wherein, the underlined sequences are the stem-loop structure sequences of PP7, showing
3 copies of PP7 are linked via a linker in series.
sgRNA-4xPP7
(SEQ ID No: 18)
NNNNNNNNNNNNNNNNNNNNGTTTGAGAGCTACTGCCATGAGGAGCAGACG
ATATGGCGTCGCTCCGATCCGACCAGCAGAGCATATGGGCTCGCTGGGGCTGCAGC
AGCAGAGGATATGGCCTCGCTGCGTGTGACAAGCAGAACATATGGGTTCGCTTGTC
ACACAAAGCAGCCAAACGGATCAAACATGGCAGTAGCAAGTTCAAATAAGGCTAG
TCCGTTATCAACTTGAAAAAGTGGCACCGAGTCGGTGCTTTTTTTATCGATGGCAA
GGGAATTC
wherein, the underlined sequences are the stem-loop structure sequences of PP7, showing
4 copies of PP7 are linked via a linker in series.
sgRNA-5xPP7
(SEQ ID No: 19)
NNNNNNNNNNNNNNNNNNNNGTTTGAGAGCTACTGCCATGAGGAGCAGACG
ATATGGCGTCGCTCCGATCCGACCAGCAGAGCATATGGGCTCGCTGGGGCTGCAGC
AGCAGAGGATATGGCCTCGCTGCGGAGTGACGAGCAGACCATATGGGGTCGCTCG
GTGTGACAAGCAGAACATATGGGTTCGCTTGTCACACAAACACTCCAAAGCAGCCA
AACGGATCAAACATGGCAGTAGCAAGTTCAAATAAGGCTAGTCCGTTATCAACTTG
AAAAAGTGGCACCGAGTCGGTGCTTTTTTTATCGATGGCAAGGGAATTC
wherein, the underlined sequences are the stem-loop structure sequences of PP7, showing
5 copies of PP7 are linked via a linker in series.
sgRNA-6xPP7
(SEQ ID No: 20)
NNNNNNNNNNNNNNNNNNNNGTTTGAGAGCTACTGCCATGAGGAGCAGACG
ATATGGCGTCGCTCCGATCCGACCAGCAGAGCATATGGGCTCGCTGGGGCTGCAGC
AGCAGAGGATATGGCCTCGCTGCGGAGTGACGAGCAGACCATATGGGGTCGCTCG
GCCACCAGAAGCAGAAGATATGGCTTCGCTTCGTGTGACAAGCAGAACATATGGG
TTCGCTTGTCACACAAAGGTGGCAAACACTCCAAAGCAGCCAAACGGATCAAACA
TGGCAGTAGCAAGTTCAAATAAGGCTAGTCCGTTATCAACTTGAAAAAGTGGCACC
GAGTCGGTGCTTTTTTTATCGATGGCAAGGGAATTC
wherein, the underlined sequences are the stem-loop structure sequences of PP7, showing
6 copies of PP7 are linked via a linker in series.
sgRNA-7xPP7
(SEQ ID No: 21)
NNNNNNNNNNNNNNNNNNNNGTTTGAGAGCTACTGCCATGAGGAGCAGACG
ATATGGCGTCGCTCCGATCCGACCAGCAGAGCATATGGGCTCGCTGGGGCTGCAGC
AGCAGAGGATATGGCCTCGCTGCGGAGTGACGAGCAGACCATATGGGGTCGCTCG
GCCACCAGAAGCAGAAGATATGGCTTCGCTTCCGGACAACTAGCAGATCATATGG
GATCGCTAGGTGTGACAAGCAGAACATATGGGTTCGCTTGTCACACAAATGTCCGA
AAGGTGGCAAACACTCCAAAGCAGCCAAACGGATCAAACATGGCAGTAGCAAGTT
CAAATAAGGCTAGTCCGTTATCAACTTGAAAAAGTGGCACCGAGTCGGTGCTTTTT
TTATCGATGGCAAGGGAATTC
wherein, the underlined sequences are the stem-loop structure sequences of PP7,
showing 7 copies of PP7 are linked via a linker in series.
sDscama30
(SEQ ID No: 24)
SPKIKPFHFTKDVQSGEREQVTCSVKLGDPPFTYTWKKDGIDINEFKDIKIEIS

In addition to the above-mentioned CRISPR FISHer systems comprising the expression vector of elements, the CRISPR FISHer system of the present invention may comprise a dCas9 protein to replace the corresponding dCas9-expressing vector form. The dCas9 protein can be obtained by transforming the corresponding dCas9-expressing vector into a host cell for recombinant expression and purification. Available host cells can include, but are not limited to: bacterial cells, fungal cells, insect cells or mammalian cells, etc., for example, commonly used E. coli cells or yeast cells, etc. In addition, dCas9 protein is also commercially available. Alternatively, the dCas9-expressing vector or dCas9 protein in the CRISPR-based imaging system of the present invention can also be replaced by a cell line stably expressing the dCas9 protein.

For example, the CRISPR FISHer system of the present invention comprises:
(1) a dCas9 protein;
(2) an engineered sgRNA-expressing vector, in which the engineered sgRNA comprises: a sgRNA backbone containing n copies of RNA aptamer, and a sgRNA sequence specific for a target gene to be detected, wherein n is an integer greater than or equal to 2;
(3) a fusion protein-expressing vector, in which the fusion protein comprises: an RNA binding motif that specifically recognizes the RNA aptamer, a multimerization peptide segment, and a fluorescent protein, which are operably linked in a manner that is not limited, and an optimal linking manner may be selected according to practical needs.

Wherein, the definition of each part of the above elements can refer to the definition described above. Specifically, the dCas9 is set forth in SEQ ID No: 1; the RNA aptamer is located in the sgRNA backbone stem-loop (i.e., sgRNA scaffold), and the RNA aptamer can be selected from, but not limited to: PP7, MS2 or BoxB, and the RNA binding motif in the fusion protein specifically recognizes the RNA aptamer in the engineered sgRNA-expressing vector, that is, the RNA aptamer and the RNA binding motif are paired, so that the RNA binding motif in the fusion protein can be, but not limited to: PCP that recognizes PP7, MCP that recognizes MS2, or N22 that recognizes BoxB; wherein the amino acid sequences of PCP, MCP, and N22 are set forth in SEQ ID No: 14, 15, and 16, respectively; in other words, in the CRISPR-based imaging system of the present invention, the RNA aptamer and the RNA binding motif exist in a paired combination, the combination is selected from: PP7 and PCP, MS2 and MCP, or BoxB and N22.
n copies of RNA aptamer mean that n copies of RNA aptamer are linked in series, which can be linked through linkers or directly. When linked through a linker, the linker can be selected from linkers commonly used in the art. Wherein n is an integer greater than or equal to 2, for example, can be 2, 3, 4, 5, 6, 7 or 8 or greater integer, its upper limit is not particularly limited, and those skilled in the art can choose a suitable value of n according to practical needs.

The multimerization peptide segment can be selected from, but not limited to, foldon trimerization small peptide, GCN4 trimerization small peptide, 3HB trimerization small peptide, 6G6H hexamerization small peptide, or sDscama30 dimerization small peptide, wherein foldon consists of an amino acid sequence as set forth in SEQ ID No: 10.

The fluorescent protein in the fusion protein can be selected from, but not limited to: green fluorescent protein (GFP), enhanced green fluorescent protein (eGFP), red fluorescent protein (RFP), blue fluorescent protein (BFP), etc.

In one embodiment, the fusion protein further comprises a nuclear localization sequence (NLS), and the nuclear localization sequence (NLS) can be located at the N-terminal or C-terminal of the fusion protein.

The CRISPR FISHer system of the present invention can realize the imaging of a single-copy gene based on aggregation of the CRISPR/fluorescent system near the gene target. For example, taking the CRISPR FISHer system of the present invention comprising PP7/PCP (as RNA aptamer and RNA binding motif, respectively) and GFP (as fluorescent protein) as an example, the aggregate formation process of the labeling and imaging is schematically illustrated as follows:
(1) Firstly, the Foldon-GFP-PCP fusion protein can spontaneously form a protein trimer (FIG. 4A), and secondly, PCP can specifically bind to PP7, that is, the Foldon-GFP-PCP fusion protein will specifically bind to the PP7 element in the engineered sgRNA.
(2) The specific aggregation process is as follows:

The sgRNA first binds to the dCas9 protein to form a complex, then the dCas9/sgRNA complex binds to the DNA sequence of the sgRNA target, and then PP7 at the sgRNA backbone stem-loop can recruit the trimerized Foldon-GFP-PCP fusion protein.

Since the trimerized Foldon-GFP-PCP fusion protein has three PCP domains, in addition to binding to PP7 on the dCas9/sgRNA complex, it can also bind to PP7 at the backbone stem-loop of other engineered sgRNAs. Other engineered sgRNAs recruit more trimerized Foldon-GFP-PCP fusion proteins. Therefore, the CRISPR FISHer system of the present invention will eventually form an aggregate of sgRNA-PP7-Foldon-GFP-PCP through repeated recruitment and combination of sgRNAs and trimerized Foldon-GFP-PCP fusion proteins. This aggregate comprises multiple GFP fluorophores, thereby achieving N-fold amplification of fluorescence signal (N is greater than or equal to 10) (FIG. 8B).
(3) Multiple sgRNAs and green fluorescent protein (GFP) will gather around the target sequence, which greatly increases the resolution and signal/background ratio of the CRISPR FISHer system, and finally achieves the effect of successful labeling and imaging of single-copy gene locus by using only one sgRNA.

In one embodiment, the amino acid sequences of the constructed Foldon-GFP-PCP and PCP-foldon-GFP fragments are as follows:

Foldon-GFP-PCP (SEQ ID No: 22, Foldon is shown in italic, GFP
is underlined by straight line, PCP is underlined by wavy line)
MGYIPEAPRDGQAYVRKDGEWVLLSTFLSGGGGSGGGGSGGGGSRKGEELFTGVVP
ILVELDGDVNGHKFSVRGEGEGDATNGKLTLKFICTTGKLPVPWPTLVTTLTYGVQCF
ARYPDHMKQHDFFKSAMPEGYVQERTISFKDDGTYKTRAEVKFEGDTLVNRIELKGID
FKEDGNILGHKLEYNFNSHNVYITADKQKNGIKANFKIRHNVEDGSVQLADHYQQNTP

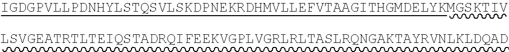

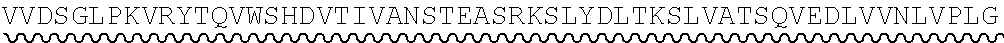

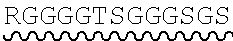

PCP-foldon-GFP (SEQ ID No: 23, Foldon is shown in italic, GFP
is underlined by straight line, PCP is underlined by wavy line)

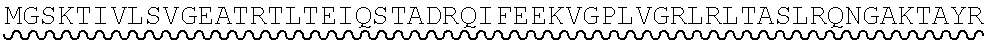

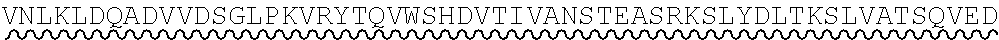

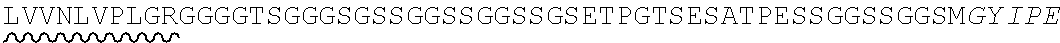

APRDGQAYVRKDGEWVLLSTFLSGGGGSGGGGSGGGGSRKGEELFTGVVPILVELDGDV
NGHKFSVRGEGEGDATNGKLTLKFICTTGKLPVPWPTLVTTLTYGVQCFARYPDHMK
QHDFFKSAMPEGYVQERTISFKDDGTYKTRAEVKFEGDTLVNRIELKGIDFKEDGNILG
HKLEYNFNSHNVYITADKQKNGIKANFKIRHNVEDGSVQLADHYQONTPIGDGPVLLP
DNHYLSTQSVLSKDPNEKRDHMVLLEFVTAAGITHGMDELYK
sDscama30-GFP-PCP (SEQ ID No: 25)
SPKIKPFHFTKDVQSGEREQVTCSVKLGDPPFTYTWKKDGIDINEFKDIKIEISSGGG
GSGGGGSGGGGSRKGEELFTGVVPILVELDGDVNGHKFSVRGEGEGDATNGKLTLKFIC
TTGKLPVPWPTLVTTLTYGVQCFARYPDHMKQHDFFKSAMPEGYVQERTISFKDDGTY
KTRAEVKFEGDTLVNRIELKGIDFKEDGNILGHKLEYNFNSHNVYITADKQKNGIKANF
KIRHNVEDGSVQLADHYQQNTPIGDGPVLLPDNHYLSTQSVLSKDPNEKRDHMVLLEF
VTAAGITHGMDELYKMGSKTIVLSVGEATRTLTEIQSTADRQIFEEKVGPLVGRLRLTAS
LRQNGAKTAYRVNLKLDQADVVDSGLPKVRYTQVWSHDVTIVANSTEASRKSLYDLT
KSLVATSQVEDLVVNLVPLGRGGGGTSGGGSGS

The CRISPR FISHer system of the present invention can greatly improve resolution and signal/background ratio (S/B ratio), and at the same time enable targeted labeling and imaging of single-copy genes.

The present invention first detects that the protein/RNA complex of dCas9, PCP-foldon-GFP and the engineered sgRNA can form an aggregate at the DNA site targeted by the sgRNA, and other combinations of RNA aptamer and RNA binding motif with similar effects can theoretically be used in the present invention as well. The above-mentioned complex with sgRNA fixedly targeting the site allows the GFP protein to aggregate at the target site, thereby achieving the purpose of visual labeling by targeting a single-copy site with a single sgRNA.

In a second aspect, the present invention provides a CRISPR-based imaging method for a target gene, the method comprising:
(i) constructing the CRISPR FISHer system described in the first aspect of the present invention, the CRISPR FISHer system comprising:
   (1) a dCas9-expressing vector;
   (2) an engineered sgRNA-expressing vector, the engineered sgRNA comprising: a sgRNA backbone containing n copies of RNA aptamer, and a sgRNA sequence specific for a target gene to be detected, wherein n is an integer greater than or equal to 2;
   (3) a fusion protein-expressing vector, the fusion protein comprising: an RNA binding motif specifically recognizing the RNA aptamer, a multimerization peptide segment and a fluorescent protein, which are operably linked in a manner that is not limited, and an optimal linking manner may be selected according to practical needs;
(ii) cell transfection: transfecting a cell to be detected with each-expressing vector in the CRISPR FISHer system;
(iii) observing aggregation spots formed by the CRISPR FISHer system by using a confocal microscope.

Wherein, the cell transfection method is a conventional transfection method that can introduce a foreign DNA sequence into the cell, comprising transfection by using a plasmid or lentivirus with the help of a transfection reagent such as LT1, Lipo2000, PEI, electroporation method, and the like.

Due to the signal gathering spots formed by the CRISPR FISHer system, the signal of the labeled target gene is enhanced, and it can be observed and photographed using a common confocal microscope in the art.

In one embodiment, in the CRISPR FISHer system, the dCas9-expressing vector can be replaced with a cell line stably expressing the dCas9 protein (e.g., a cell line transfected with the dCas9-expressing vector). The dCas9 is set forth in SEQ ID No: 1.

In one embodiment, the CRISPR FISHer system may comprise a dCas9 protein to replace the corresponding dCas9-expressing vector form. For example, in the CRISPR FISHer system of the present invention, the dCas9 protein-expressing vector can be replaced with a dCas9 protein. The dCas9 protein or fusion protein can be obtained by transforming the corresponding expression vector into a host cell for recombinant expression and purification. Available host cells may include, but are not limited to: bacterial cells, fungal cells, insect cells or mammalian cells etc., for example, commonly used E. coli cells or yeast cells, etc. In addition, the dCas9 protein is also commercially available.

For example, the CRISPR FISHer system of the present invention comprises:
(1) a dCas9 protein;
(2) an engineered sgRNA-expressing vector, the engineered sgRNA comprising: a sgRNA backbone comprising n copies of RNA aptamer, and a sgRNA sequence specific for a target gene to be detected, wherein n is an integer greater than or equal to 2;
(3) a fusion protein-expressing vector, the fusion protein comprising: an RNA binding motif that specifically recognizes the RNA aptamer, a multimerization peptide segment, and a fluorescent protein, which is operably linked in a manner that is not limited, and the best connection manner can be selected according to practical needs.

Wherein, the definition of each element is referred to the definition of each element in the first aspect herein.

In one embodiment, the fusion protein further comprises a nuclear localization sequence (NLS), and the nuclear localization sequence (NLS) can be located at the N-terminal or C-terminal of the fusion protein.

When the CRISPR FISHer system comprises a dCas9 element in protein form, the target gene imaging method comprises the following steps:
(i) cell transfection: transfecting (for example, by electroporation) the dCas9 protein, sgRNA-expressing vector and fusion protein-expressing vector contained in the CRISPR FISHer system into cells to be detected;
(ii) observing aggregation spots formed by the CRISPR FISHer system using a confocal microscope.

In one embodiment, the CRISPR-based gene imaging method described in the present invention can be used for labeling and imaging a single-copy gene in a living cell.

In one embodiment, the CRISPR-based gene imaging method described in the present invention can be used for labeling and imaging a multi-copy gene in a living cell.

In one embodiment, the CRISPR-based gene imaging method described in the present invention can be used for labeling and imaging a non-repetitive sequence in chromosomal DNA or extra-chromosomal DNA in a living cell.

In one embodiment, the CRISPR-based gene imaging method described in the present invention can be used for labeling and imaging an extrachromatin circular DNA element (eccDNA) in a living cell.

In one embodiment, the CRISPR-based gene imaging method described in the present invention can be used for regional labeling and imaging of a CRISPR binding site, not limited to a genome, for example, an extrachromatincircular DNA (eccDNA), exogenously expressed plasmid, HBV gene sequence, and double-stranded AAV DNA of adeno-associated virus (AAV) may also be clearly imaged.

In a third aspect, the present invention provides a kit for CRISPR-based gene labeling and imaging, the kit comprising:
(1) a dCas9-expressing vector;
(2) an engineered sgRNA-expressing vector, the engineered sgRNA comprising: a sgRNA backbone comprising n copies of RNA aptamer, and a sgRNA sequence specific for a target gene to be detected, wherein n is an integer greater than or equal to 2;
(3) a fusion protein-expressing vector, the fusion protein comprising: an RNA binding motif that specifically recognizes the RNA aptamer, a multimerization peptide segment, and a fluorescent protein, which is operably linked in an order that is not limited, and an optimal linking order may be selected according to practical needs;
wherein, the dCas9-expressing vector, the engineered sgRNA-expressing vector and the fusion protein-expressing vector are each stored in a separate container.

In one embodiment, the dCas9-expressing vector can be replaced with a cell line stably expressing the dCas9 protein.

In one embodiment, the kit may comprise a dCas9 protein in place of the corresponding dCas9-expressing vector form. For example, the kit comprises:
(1) a dCas9 protein;
(2) an engineered sgRNA-expressing vector, the engineered sgRNA comprising: a sgRNA backbone comprising n copies of RNA aptamer, and a sgRNA sequence specific for a target gene to be detected, wherein n is an integer greater than or equal to 2;
(3) a fusion protein-expressing vector, the fusion protein comprising: an RNA binding motif that specifically recognizes the RNA aptamer, a multimerization peptide segment, and a fluorescent protein, which are operably linked in a manner that is not limited, and an optimal linking manner may be selected according to practical needs;
wherein the dCas9 protein, the engineered sgRNA-expressing vector and the fusion protein-expressing vector are each stored in a separate container.

In one embodiment, the engineered sgRNA-expressing vector is driven by a U6 promoter, which may be a mouse U6 promoter (mU6) or a human U6 promoter (hU6);
the RNA aptamer is located in the sgRNA backbone stem-loop (i.e., sgRNA scaffold), and the RNA aptamer can be selected from, but not limited to: PP7, MS2 or BoxB;
n copies of RNA aptamer represent that n copies of RNA aptamer are linked in series, which can be linked through a linker or directly, and the linker can be selected from linkers commonly used in the art, wherein n is an integer greater than or equal to 2, for example, can be an integer of 2, 3, 4, 5, 6, 7 or 8 or greater, its upper limit is not particularly limited, and those skilled in the art can select a suitable value of n according to practical needs;
the multimerization peptide segment can be selected from, but not limited to, foldon trimerization small peptide, GCN4 trimerization small peptide, 3HB trimerization small peptide 6G6H hexamerization small peptide, or sDscama30 dimerization small peptide, wherein foldon consists of an amino acid sequence as set forth SEQ ID No: 10;
wherein, the RNA binding motif in the fusion protein specifically recognizes the RNA aptamer in the engineered sgRNA, that is, the RNA aptamer and the RNA binding motif are paired, so that the RNA binding motif in the fusion protein can be, but not limited to: PCP that recognizes PP7, MCP that recognizes MS2, or N22 that recognizes BoxB; wherein the amino acid sequences of PCP, MCP and N22 are set forth in SEQ ID No: 14, 15, and 16, respectively; in other words, in the CRISPR-based imaging system of the present invention, the RNA aptamer and the RNA binding motif exist in a paired combination, and the combination is selected from the group consisting of: PP7 and PCP, MS2 and MCP or BoxB and N22.

The fluorescent protein in the fusion protein can be selected from, but not limited to: green fluorescent protein (GFP), enhanced green fluorescent protein (EGFP), red fluorescent protein (RFP), blue fluorescent protein (BFP), etc.

In one embodiment, the fusion protein further comprises a nuclear localization sequence (NLS), and the nuclear localization sequence (NLS) can be located at the N-terminal or C-terminal of the fusion protein.

The above contents are a summary and thus simplifications, generalizations and omissions of detail have been included where necessary. Accordingly, those skilled in the art will recognize that this summary is illustrative only and is not intended to be limiting in any way. Other aspects, features and advantages of the methods, editing libraries and/or other subject matters described herein will become apparent from the teachings presented herein. The summary is provided to introduce a simplified introduction to a selection of concepts that are further described below in the detailed description. This summary is not intended to identify key features or essential features of the claimed subject matter, nor is it intended to be used to determine the scope of the claimed subject matter. Furthermore, the contents of all references, patents, and published patent applications cited throughout the present application are hereby incorporated by reference in their entirety.

### BRIEF DESCRIPTION OF DRAWINGS

By referring to the following drawings, those skilled in the art will more easily understand the technical solution of the present invention. These drawings form a part of the present invention.

FIG. 1 shows the fluorescence of the fusion construct of foldon element and GFP expressed in 293T cells for 12 hours: fluorescence of foldon-GFP or GFP-foldon in 293T cells 12 hours after transfection. It can be seen that whether in the control group (left column, only GFP) or the experimental groups (middle column and right column, foldon was fused to the N-terminal or C-terminal of GFP, respectively, and GGS schematically indicates the linker sequence), after transfection of 12 hours, the fluorescence intensity had reached a near-saturation state.

FIG. 2 shows the western blot native (i.e., non-denaturing) gel detection results of GFP. Wherein, GGS schematically represents a linker sequence. It can be seen that compared with the GFP of the control group (wild type, left lane), the trimerization of GFP occurred no matter whether the foldon element was fused at the N-terminal (middle lane) or the C-terminal (right lane) of GFP, but the trimerization effect of the fusion of foldon at the N-terminal of GFP is stronger than that of the fusion at the C-terminal.

FIG. 3 shows a schematic diagram of the structure and a schematic diagram of the function mode of each element of one of the CRISPR FISHer system versions (dCas9, sgRNA-8×PP7, PCP-foldon-GFP) prepared in Example 1 of the present invention.

FIG. 4 shows purified proteins PCP-GFP and foldon-GFP-PCP separated by SDS-PAGE gel (A, denaturing conditions) and native gel (B, non-denaturing conditions), and the results show: the trimerization occurred in foldon-GFP-PCP compared with the control of PCP-GFP (B); representative photomicrographs for PCP-GFP and foldon-GFP-PCP each incubating with a series of sgRNAs (including normal sgRNA (i.e., not containing PP7) or engineered sgRNA containing n copies of PP7, n was an integer from 1 to 8) (C), and in this assay, the concentrations of PCP-GFP, foldon-GFP-PCP and sgRNA were 1 μM, 1 μM, and 0.5 μM, respectively, the area for each field was 1695 μm²; the statistical distribution of individual aggregates (GFP dots) per 15250 μm² after incubation at room temperature (D); and the schematic diagram of proposed assembly model PCP-GFP or foldon-GFP-PCP with sgRNAs and engineered sgRNA with PP7 aptamers (E).

FIG. 5 shows that Foldon-GFP-PCP allowed the CRISPR FISHer system to achieve robust genomic locus tracking with improved signal/background ratio (S/B ratio).
(A) shows the schematic diagram of the aggregation process of the CRISPR FISHer system (in a version comprising dCas9, sgRNA-2×PP7 and Foldon-GFP-PCP) at the target site. It shows the schematic of CRISPR FISHer being recruited to the target site. The sgRNA first binds to the dCas9 protein to form a complex, then the dCas9/sgRNA complex binds to the DNA sequence of the sgRNA target, and then the exposed PP7 sequence on sgChr3Rep-2×PP7 recruits the trimerized Foldon-GFP-PCP fusion protein, which assemblies and aggregates at Chr3q29 (about 500 copies, termed as Chr3Rep).
(B) shows enrichment of foldon-GFP-PCP at the Chr3Rep loci (arrows) labeled by dCas9-mCherry in live U2OS cell. White arrows indicate the Chr3Rep gene locus. Fluorescent imaging results showed that Foldon-GFP-PCP aggregation spots appeared 4 hours after transfection, which co-localized with the Chr3Rep gene locus and gradually became brighter and clearer. This result indicates that target DNA-bound dCas9/sgChr3Rep recruited foldon-GFP-PCP to the targeted gene locus, and simultaneously enhanced the GFP signal at the target site and reduced non-specific background.
(C) shows the colocalization of foldon-GFP-PCP (green) and dCas9-mCherry (red) on the Chr3Rep locus in U2OS cells, HeLa cells and HepG2 cells co-transfected with the foldon-GFP-PCP-expressing vector, the dCas9-mCherry-expressing vector and the sgChr3Rep-2×PP7-expressing vector. Co-localization was detected 24 hours after the transfection, indicating that foldon-GFP-PCP co-localized with dCas9-mCherry 24 hours after the transfection. BFP: used as an indicator of the nuclei and sgRNA-2×PP7 expression.
(D) shows the comparison of foldon-GFP-PCP, PCP-GFP and dCas9-EGFP labeling of telomere loci in U2OS cells. sgGal4 is used as the negative control. The dotted lines (up) label area used to generate respective line scans (down). Middle: the spatial distribution of telomere loci.
(E and F) show the comparison of the signal/background ratio (S/B ratio) of the telomere loci labeled with foldon-GFP-PCP, PCP-GFP, and dCas9-EGFP.
(E) shows the data presented as mean±SEM: dCas9-EGFP (2.056±0.385, n=21), PCP-GFP (1.849±0.385, n=20), foldon-GFP-PCP (18.579±4.515, n=23). The S/B ratios of the experimental group could be up to 10 times that of the control group.
(F) shows the S/B enhancement based on the signal/background ratio (S/B) in (E).

In this version of the CRISPR FISHer system, n.s. indicates non-significant, and *** indicates P<0.001 (Wilcoxon test). Scale bar is 5 μm.

FIG. 6 shows the GFP fluorescence imaging results (A) and fluorescence intensity (B) of the experimental group (with foldon) and the control group (without foldon) in telomere labeling under the same transfection conditions, and the 3D imaging results of the cells in the experimental group (C).
(A) shows the fluorescence of the fusion construct of foldon and GFP targeting telomere repetitive sequence in 293T cells after 12 hours of expression. It can be seen that the fluorescence intensity of the group containing PCP-foldon-GFP was significantly higher than that of the control group (PCP-GFP). The sgRNA targeting telomeres comprised 8×PP7 (sgTelomere-8×PP7); sgNT had no targeting sequence and thus could not be located on the chromosome.
(B) shows the comparison of fluorescence intensity values of representative targeted loci.
(C) shows the 3D imaging results of the cells in the experimental group.

FIG. 7 shows the GFP fluorescence detection results of the single-copy gene TOP 3 labeled in the experimental groups and the control groups under the same transfection conditions. The first two columns from the left are the experimental groups, in which dCas9, sgTOP3-8×PP7 and PCP-foldon-GFP were expressed, and the CRISPR FISHer system was used to label the position of the single-copy gene TOP3 when the chromosome was replicated and not replicated. In the fifth column, on the basis of the first two columns, a sequence from the TOP3 gene was exogenously transferred as the targeting sequence of the sgRNA, and it could be seen that the signal dots of green fluorescence increased significantly. The third column and the fourth column are the control groups of the fifth column, in which dCas9, sgTOP3-8×PP7, PCP-foldon-GFP and empty T vector (T vector) were expressed. The last column used a system expressing dCas9, sgTOP3-8×PP7 and PCP-GFP as control, indicating that the CRISPR FISHer system could achieve highly sensitive labeling and imaging of single-copy genes compared to the existing system.

FIG. 8 shows that the Foldon-GFP-PCP-based CRISPR FISHer system could achieve the labeling and imaging of non-repetitive sequences in chromosomal DNA or extra-chromosomal DNA.
(A) shows the labeling and imaging results of the non-repetitive region of the PPP1R2 single-copy gene in U2OS cells, in which the upper row shows the representative images of PPPIR2 labeled in the PCP-GFP group (diffuse green fluorescent signal) and the Foldon-GFP-PCP group (2-4 green fluorescence signal dots), respectively; and the lower row shows the distribution of the representative PPP1R2 loci in the upper row in the z-section.
(B) shows the simulation diagram of the CRISPR FISHer system with sgRNA-2×PP7 when targeting a gene locus.
(C) shows the schematic diagram of dual-color CRISPR imaging for loci PPP1R2 (GFP) and chromosome 3 repetitive region (Chr3Rep) (tdTomato) in U2OS cells. The distance between the Chr3Rep and the non-repetitive PPPIR2 site is about 15 kb.
(D and E) show the comparison of CRISPR FISHer and conventional CRISPR-Sirius labeling for the single-copy gene PPP1R2 (green signal). sgPPP1R2.1-2×PP7 or sgPPP1R2.1-8×PP7 were used to target the PPPIR2 gene. In (D), red-labeled Chr3Rep served as an internal control, and its imaging system comprised Chr3Rep-2×MS2, dCas9 and stdMCP-tdTomato; the fusion of BFP with NLS indicated the nuclei and sgRNA-PP7 transfection. The dotted line on the left indicates the area producing the fluorescence intensity value on the right. (E) Comparison of signal/background ratio between the CRISPR-based FISHer (Foldon-GFP-PCP) and conventional CRISPR-Sirius (PCP-GFP). The T-test showed that the signal/background ratio of Foldon-GFP-PCP in labeling the single-copy gene was significantly higher than that of PCP-GFP (P<0.001***).
(F and G) show the three-color CRISPR imaging for loci of the PPPIR2 gene (green), Chr3Rep (red) and Chr13Rep (purple) in U2OS cells. (F) shows the schematic diagram of the target loci on Chr3 and Chr13. (G) shows in situ imaging for PPP1R2 gene (green, foldon-GFP-PCP), Chr3Rep (red, stdMCP-tdTomato), and Chr13Rep (purple, N22-Halo). In the fluorescent labeling image, the dotted line on the left indicated the area producing the fluorescence intensity value on the right. It is an.
(H and I) show that the labeling and imaging of single-copy genes TOP3 or TOP1 in U2OS cells using the CRISPR FISHer. The stdMCP-tdTomato-labeled Chr3Rep (red) served as an internal control. TOP 3 was located on chromosome 17, and TOPI was located on chromosome 21. (H) shows the schematic diagram of target loci on Chr3 and Chr17 or Chr20. (I) shows images for TOP3 or TOP1 gene (green, foldon-GFP-PCP) and Chr3Rep (red, stdMCP-tdTomato, internal control). In the fluorescence labeling image, the dotted line on the left indicated the area producing the fluorescence intensity value on the right, the dotted line runs through the selected red and green fluorescence signal dots, and the right side corresponds to its fluorescence intensity value.
(J) shows that the CRISPR FISHer system was used to detect the HBV integration into the genome in the Hep3B cell line. sgGal4 served as an internal control (diffuse green fluorescence signal), and the CRISPR FISHer system with sgHBV targeting the S protein of HBV showed green dots, indicating the presence of HBV virus in Hep3B cells.
(K) shows the number of green fluorescence signal dots counted in 30 Hep3B cells, representing the copy number of HBV loci in Hep3B cells (n=30).

FIG. 9 shows that the CRISPR FISHer system tracked CRISPR-induced DNA double-strand breakage (DSB) and non-homologous end-joining repair.
(A) shows the schematic diagram of intrachromosomal separation and rejoining through labeling two-ended DSB fragments after DSB induction. First, the CRISPR-Sirius system was used to label the repetitive sequence region of chromosome 3 (Chr3Rep, red), and the CRISPR FISHer was used to label the PPPIR2 gene (green). 16 hours after delivering DNA loci labeling systems, SaCas9 and its corresponding sgRNA (cutting the middle region between the red and green labeling sites) were delivered by nucleofection for inducing DSB between the two labeled loci.
(B) shows the representative fluorescent imaging of DSB-induced intrachromosomal dissociation and rejoining in a single cell. White box showing different DNA loci.
(C and D) show the time-lapse imaging and quantified distance of DNA loci pair 1 in (B). It can be seen that the red signal dots and the green signal dots separated at 60 min, and then gradually approached and finally completely overlapped, indicating the process of chromosome dissociation and re-repair.
(E and F) show the time-lapse imaging and quantified distance of DNA loci pairs 2 and 3 in (B) at different time points. It can be seen that after the dissociation and repair at each of the above 2 loci, the interchromosomal rejoining gradually appeared.
(G) shows the schematic diagram of DSB induced interchromosomal translocation between Chr3 and Chr13. Its labeling strategy was similar to FIG. 8F. SaCas9/sgRNA was delivered to produce DNA cutting between the labeled loci on Chr3 and SPACA7 gene on Chr13 (delivered 16 h after labeling system delivery).
(H) shows the time-lapse images of labeling and imaging fluorescence showing intrachromosomal dissociation and interchromosomal translocation between Chr3 and Chr13. Colored arrows indicate three DNA loci for tracking (green, PPP1R2; red, Chr3Rep; purple, Chr13Rep). The white box showed a local enlargement. Time-lapse imaging started from 4 hours post saCas9/sgRNA delivery. It can be seen that the separation of the red and green fluorescence signal dots within the first 60 minutes indicated that the SaCas9 targeting chromosome 3 had been cut, and the complete overlapping between the green fluorescence signal and pink fluorescence signal at 75 minutes indicated the translocation between the long arm of chromosome 3 and the short arm of chromosome 13 occurred at this time.
(I) shows the distance of the DNA loci pairs in (H). The red line indicated the distance between Chr3Rep (red) and PPP 1R2 (green) paired foci; and the purple line indicated the distance between Chr13Rep (purple) and PPP1R2 (green) paired foci.

FIG. 10 shows that the CRISPR FISHer is capable of tracking the dynamic location of extrachromosomal DNA in living cells in real time.
(A) shows the strategy flow for identifying eccDNA from HepG2.
(B) shows the junctional sequence information of three representative eccDNAs identified in HepG2 cells.
(C) shows the schematic strategy of the eccDNA labeling by using CRISPR FISHer. sgRNA target sites located at junction regions of eccDNAs
(D) shows the representative images of the eccDNA labeled with CRISPR FISHer. sgGal4 served as a control sgRNA, and presented a diffuse green fluorescent signal.
(E) shows the statistical results of four kinds of eccDNAs in HepG2 cells.
(F) shows the motion trajectory diagram of Chr3Rep, PPPIR2 and eccBEND3 during a 5-min period.
(G) shows the statistic results of the trajectory lengths of Chr3Rep, PPPIR2 and eccBEND3 during a 5-min period, in which the T-test showed that the motion trajectory length of eccDNA was significantly increased as compared with those of the chromosome and the gene on chromosome (P<0.001***). It can be seen that eccDNA, as an extrachromosomal DNA, has a great difference in its movement mode from the chromosome and the gene on chromosome, the difference may be associated with its specific physiological functions.
(H) shows the amplification and labeling strategy for linearized eccDNA. Dotted box indicting the CRISPR FISHer targeting locus as well as junction regions of eccDNA.
(I) shows the motion trajectories of linearized eccBEND3, eccPRKCB, and eccGABRR1 during a 5-min period.
(J) shows the statistical graph of the comparison of trajectory lengths between circular eccDNA and linearized eccDNA during a 5-min period.
(K) shows the schematic labeling strategy of eccDNA (e.g., adeno-associated virus (AAV)) by using CRISPR FISHer.
(L and M) show the double-stranded (ds) adeno-associated virus (AAV) DNA loci in nuclei labeled with CRISPR FISHer in U2OS cells. (L) The appearance and increasing formation of ds AAV DNA foci over time were shown in a single live cell. The sgRNA targeting mouse TBG carried by AAV was used. 1 (M) shows that in a single living cell, double-stranded AAV DNA fluorescently labeled spots appeared and gradually increased over time.
(N) shows the motion trajectory of AAV in U2OS cell nuclei during a 5-min period.
(O) shows the statistics of the motion trajectory length of AAV in U2OS cell nuclei during a 5-min period.

FIG. 11 shows that the trimeric foldon-GFP-PCP enables the CRISPR FISHer system to label repetitive sequences in a variety of cell lines.
(A) The dual-color CRISPR imaging shows the co-localization of foldon-GFP-PCP (green) and dCas9-mCherry (red) appeared at the multi-copy locus Chr13Rep in U2OS, HeLa and HepG2 cells. Scale bar is 5 μm.
(B) The representative single-layer diagram of the z-axis scanning of telomere imaging in FIG. 5D.

FIG. 12 shows the distribution of repetitive sequences on different chromosomes in the human genome.

FIG. 13 shows the signal characteristics of foldon-GFP-PCP (green) in different control groups under diverse transfection conditions. The upper row shows the image of the foldon-GFP-PCP green channel superimposed with the Hoechest blue channel, and the middle and lower rows show the images of the green channel and the blue channel, respectively. From left to right, the first column shows the transfection with plasmids expressing foldon-GFP-PCP; the second column shows the transfection with plasmids expressing normal sgPPP1R2.1 and foldon-GFP-PCP; the third column shows the transfection with plasmids expressing sgPPP1R2.1-2×PP7 and foldon-GFP-PCP; the fourth column shows the transfection with plasmids expressing foldon-GFP-PCP and dCas9; the fifth column shows the transfection with plasmids expressing normal sgPPP1R2.1, foldon-GFP-PCP and dCas9; the sixth column shows the transfection with plasmids expressing SgGal4-2×PP7 which has no target sequence in cells. Hoechest was used to stain the nuclei. Scale bar is 5 μm.

FIG. 14 shows that CRISPR FISHer enables visualization of nonrepetitive sequences in the PPPIR2 gene in live U2OS cells.
(A) shows the schematic diagram that the co-localization of the single-copy gene locus PPPIR2 and the multi-copy gene locus Chr3Rep that were labeled by the dual-color CRISPR system CRISPR-FISHer (green) and CRISPR-Sirius (red) in U2OS cells.
(B) shows the result diagrams of the co-localization of the single-copy gene locus PPPIR2 labeled using CRISPR-FISHer (green) in combination with sgRNA containing 2×PP7 and 8×PP7 and the repetitive sequence locus Chr3Rep labeled by using CRISPR-Sirius (red).
(C) shows the result diagrams of the single-copy gene locus PPPIR2 labeled by ng the CRISPR-Sirius (PCP-GFP) system combined with sgRNA containing 8×PP7. Scale bar is 5 μm.

FIG. 15 shows the result diagrams of the single-copy gene locus PPPIR2 labeled by the CRISPR FISHer (green) system in Hela and HepG2 cells. CRISPR-Sirius (red) was used to label the repetitive sequence locus Chr3Rep. Scale bar is 5 μm.

FIG. 16 shows that the CRISPR FISHer (green) system enables labeling of non-repetitive loci in cells.
(A) shows the schematic diagram of the co-localization of the single-copy gene locus SOX1 and the multi-copy locus Chr13Rep that were labeled by the dual-color CRISPR system CRISPR-FISHer (green) and CRISPR-Sirius (red) in U2OS cells.
(B) shows the result diagram of the co-localization of the single-copy gene locus SOX1 labeled by using CRISPR FISHer (green) in combination with the sgRNA containing 2×PP7 and 8×PP7 and the repetitive sequence locus Chr13Rep labeled by using CRISPR-Sirius (red).
(C) shows the result diagrams of the single-copy gene locus SOX1 labeled using the CRISPR-Sirius (PCP-GFP) system combined with sgRNA containing 8×PP7. Scale bar is 5 μm.
(D and E) show the schematic diagrams and result diagrams of the single-copy gene loci (TOP 3, TOP1) and multi-copy loci (Chr3Rep, Chr13Rep) by using the dual-color CRISPR system CRISPR-FISHer (green) and CRISPR-Sirius (red) in U2OS cells.
(F and G) show the results of the PCR amplification and sequencing of HBV gene fragments in HepG2, Huh7 and Hep3B cells.

FIG. 17 shows the dynamic process of non-homologous end-joining after DNA breakage in U2OS cells, visualized using CRISPR FISHer (green) and CRISPR-Sirius (red).
(A) shows the schematic diagram of co-labeling PPPIR2 and repetitive sequence locus Chr3Rep in U2OS cells using CRISPR FISHer (green) and CRISPR-Sirius (red).
(B and C) show the time-lapse imaging of the dynamic process of non-homologous end-joining after DNA breakage, after co-labeling of PPPIR2 and repetitive sequence locus Chr3Rep in U2OS cells using CRISPR-FISHer (green) and CRISPR-Sirius (red).

FIG. 18 shows the identification results of genome sequences after chromosomal rejoining.
(A) shows the schematic diagram of genome sequence assembly after chromosomal rejoining.
(B and C) show the Sanger sequencing results of genome sequences after chromosomal rejoining.

FIG. 19 shows the results of identifying eccDNA and tracking eccDNA movement in real time in HepG2 cells.
(A) shows the position information and sizes of multiple eccDNA fragments identified in HepG2 cells.
(B and C) show the strategy and result diagrams of identifying eccDNA sequences in HepG2 cells by three rounds of PCR.
(D) shows the trajectories of circular eccDNA and Chr13 labeled using the CRISPR FISHer system.

FIG. 20 shows the results of the CRISPR FISHer system comprising sDscama30-GFP-PCP (green) and dCas9-mCherry (red).
(A) Representative images showing colocalization of sDscama30-GFP-PCP (green) and dCas9-mCherry (red) on the Telomere and Chr3Rep locus in U2OS cells. The plasmids expressing sDscama30-GFP-PCP, dCas9-mCherry, sgChr3Rep-3×PP7, and BFP were co-transfected. BFP: used as an indicator of the nuclei and sgRNA-3×PP7 expression.
(B) Comparison of sDscama30-GFP-PCP and PCP-GFP labeling of single-copy gene PPPIR2. sgPPP1R2.1-7×PP7 was used for targeting the PPPIR2 gene (green); sgChr3Rep-2×MS2 was used for labeling Chr3Rep loci (red, internal control).

### DETAILED DESCRIPTION OF INVENTION

While the present invention may be embodied in many different forms, disclosed herein are specific illustrative embodiments thereof that demonstrate the principles of the present invention. It should be emphasized that the present invention is not limited to the particular embodiments illustrated herein. Furthermore, any section headings used herein are for organizational purposes only and are not to be construed as limiting the described subject matter.

Unless otherwise defined herein, scientific and technical terms used in connection with the present invention shall have the meanings commonly understood by those of ordinary skills in the art. Further, unless otherwise required in the context, terms in the singular shall include the plural, and terms in the plural shall include the singular. More specifically, as used in this description and the appended claims, the singular forms “a,” “an,” and “the” include plural referents unless the context clearly dictates otherwise. In the present application, the use of “or” means “and/or” unless stated otherwise. Furthermore, the use of the term “comprising” as well as other forms such as “comprise” and “comprises” is not limiting. Furthermore, the ranges provided in the description and the appended claims include all values between the endpoints and breakpoints.

### Definition

To better understand the present invention, definitions and explanations of related terms are provided below.

The term CRISPR (Clustered regularly interspaced short palindromic repeats) is a repetitive sequence in the genome of prokaryotic organisms. It is an immune weapon produced in the combat between bacteria and viruses in the history of life evolution. In short, during the infection with a virus, the virus can integrate its genes to the bacterial genome, and use the bacterial cell tools to serve its gene replication. However, in order to clear the foreign invasion genes of the virus, the bacteria have evolved the CRISPR-Cas9 system. Using this system, the bacteria can quietly excise the integrated viral genes from their own chromosomes, and this is the unique immune system of bacteria. Discovered in the early 1990s, CRISPR technique quickly became the most popular gene-editing tool in the fields of human biology, agriculture, and microbiology as research seeped in.

In general, “CRISPR system” collectively refers to transcripts and other elements involved in the expression of or directing activity CRISPR-associated (abbreviated as “Cas”) genes, including sequences encoding a Cas gene, a tracr (trans-activating CRISPR) sequence (e.g., a tracrRNA or an active partial tracrRNA), a tracr-mate sequence (encompassing a “direct repeats” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system are derived from a Type I, Type II, or Type III CRISPR system. In some embodiments, one or more elements of a CRISPR system are derived from a particular organism comprising an endogenous CRISPR system, such as Streptococcus pyogenes. In general, a CRISPR system is characterized by elements that promote the formation of the CRISPR complex (also referred to as a protospacer in the context of an endogenous CRISPR system) at the site of the target sequence. In the context of formation of a CRISPR complex, “target sequence” refers to a sequence to which a guide sequence is designed to have complementarity, where hybridization between the target sequence and the guide sequence promotes the formation of a CRISPR complex. Full complementarity is not necessarily required, provided that there is sufficient complementarity to cause hybridization and promote the formation of a CRISPR complex. A target sequence can comprise any polynucleotide, such as DNA or RNA polynucleotides. In some embodiments, the target sequence is located in the nucleus or cytoplasm of a cell. In some embodiments, the target sequence may be located in an organelle of a eukaryotic cell, for example, mitochondria or chloroplast. A sequence or template that may be used for recombination into the targeted locus comprising the target sequence is referred to as an “editing template” or “editing polynucleotide” or “editing sequence”. In the present invention, an exogenous template polynucleotide may be referred to as an editing template. In one aspect of the present invention, the recombination is homologous recombination.

Cas refers to a CRISPR-associated (abbreviated as “Cas”) gene, and can also be used to refer to an expression product of the gene (called CRISPR enzyme or Cas9 enzyme). The currently discovered Cas includes Cas1 to Cas10 and other types. Cas genes have co-evolved with CRISPR and together constitute a highly conserved system.

dCas9 refers to “dead Cas9”, i.e., Cas9 without DNA cleavage catalytic activity (e.g., by mutating D10A and H840A), and usually a Cas protein with one or more NLS intranuclear localization information or a fusion protein containing Cas protein.

“sgRNA”: a guide RNA that binds to Cas9 (or dCas9). The sgRNA used in the present system also carries an RNA aptamer that binds to an RNA binding motif, such as PP7, MS2 or BoxB. PP7: a binding region of other RNA binding motifs other than Cas9 (or dCas9) fused with guide RNA (sgRNA), which generally binds PCP.

PCP: a phage coat-binding motif that recognizes PP7.

Foldon: a short peptide derived from the C-terminus of T4 bacteriophage fibritin, and this domain is composed of three identical subunits, and each subunit includes a β-hairpin structure. After fusing foldon with a target protein, it can make the target protein spontaneously forms a trimer (A. V. Letarov et al., Biochemistry (Moscow), Vol. 64, No. 7, 1999, pp. 817-823. Translated from Biokhimiya, Vol. 64, No. 7, 1999, pp. 974-981).

“CRISPR-Sirius Imaging System” is a CRISPR-based imaging system developed by Ma Hanhui et al. in 2018. The system consists of three parts: the first part is a vector expressing dCas9, the second part is a vector expressing sgRNA-8×MS2/PP7, and the third part is a vector expressing MCP/PCP-fluorescent protein. When the above three vectors are co-transfected into a cell, the fluorescent protein can form a sgRNA-fluorescent protein complex through the binding between MS2 or PP7 and MCP or PCP, and the sgRNA-fluorescent protein complex will recognize a certain site in the genome and guide dCas9 to bind at the corresponding site, so as to realize the labeling and imaging of the site. Due to the presence of stable 8×MS2/PP7, 8 fluorescent proteins will also be stably aggregated, so that the resolution of the imaging system is greatly improved by this method. The imaging resolution limit of the system reaches up to 22 copies, however, gene loci below 22 copies are impossible to observe through the system.

The terms “polynucleotide”, “nucleotide”, “nucleotide sequence”, “nucleic acid” and “oligonucleotide” are used interchangeably. They refer to a polymeric form of nucleotides, either deoxyribonucleotides or ribonucleotides, or analogs thereof, in any length. A polynucleotide can have any three-dimensional structure and can perform any function, known or unknown. The following are non-limiting examples of polynucleotide: coding or non-coding region of a gene or gene fragment, multiple loci (one locus) defined by junctional analysis, exon, intron, messenger RNA (mRNA), transfer RNA, ribosomal RNA, short hairpin RNA (shRNA), micro-RNA (miRNA), ribozyme, cDNA, recombinant polynucleotide, branched polynucleotide, plasmid, vector, isolated DNA of any sequence, isolated RNA of any sequence, nucleic acid probe, and primer. A polynucleotide may comprise one or more modified nucleotides, such as methylated nucleotides and nucleotide analogs. Modification(s), if present, may be made to nucleotide structure before or after polymer assembly. The sequence of nucleotides may be interrupted by non-nucleotide components. The polynucleotide can be further modified after polymerization, such as by conjugation with labeled components.

“Complementarity” refers to the ability of a nucleic acid to form one or more hydrogen bonds with another nucleic acid sequence by means of traditional Watson-Crick or other non-traditional types. Percent complementarity represents the percentage of residues in a nucleic acid molecule that can form hydrogen bonds (e.g., Watson-Crick base pairing) with a second nucleic acid sequence (e.g., 5, 6, 7, 8, 9, 10 out of 10 are 50%, 60%, 70%, 80%, 90%, and 100% complementary). “Complete complementary” means that all contiguous residues of one nucleic acid sequence form hydrogen bonds with the same number of contiguous residues in a second nucleic acid sequence. “Substantially complementary” as used herein refers to a complementary degree of at least 60%, 65%, 70%, 75%, 80%, 85%, 90%, 95%, 97%, 98%, 99%, or 100% on a region having 8, 9, 10, 11, 12, 13, 14, 15, 16, 17, 18, 19, 20, 21, 22, 23, 24, 25, 30, 35, 40, 45, 50 or more nucleotides, or refers to two nucleic acids that hybridize under stringent conditions.

“Expression” as used herein refers to a process by which a polynucleotide (e.g., mRNA or other RNA transcript) is transcribed from a DNA template and/or a process by which the transcribed mRNA is subsequently translated into a peptide, polypeptide or protein. The transcript and encoded polypeptide may be collectively referred to as “gene product.” If the polynucleotide is derived from a genomic DNA, the expression may comprise splicing mRNA in an eukaryotic cell.

Generally, and throughout the present description, the term “vector” refers to a nucleic acid molecule capable of delivering another nucleic acid molecule to which it has been linked. Vectors include, but are not limited to, nucleic acid molecules that are single-stranded, double-stranded, or partially double-stranded; nucleic acid molecules that include one or more free ends, no free ends (e.g., circular); nucleic acid molecules that include DNA, RNA, or both; and other miscellaneous polynucleotides known in the art. One type of vector is a “plasmid”, which refers to a circular double-stranded DNA loop into which an additional DNA segment can be inserted, for example, by a standard molecular cloning technique. Another type of vector is a viral vector, in which a virus-derived DNA or RNA sequence is present in a vector for packaging a virus (e.g., retrovirus, replication defective retrovirus, adenovirus, replication defective adenovirus, and adeno-associated virus). Viral vector also comprises a polynucleotide carried by a virus used for transfection into a host cell. Certain vectors (e.g., bacterial vectors with a bacterial replication origin and episomal mammalian vectors) are capable of autonomous replication in the host cell into which they are introduced. Other vectors (e.g., non-episomal mammalian vectors) are integrated into the genome of the host cell upon introduction into the host cell and thereby replicate along with the host genome. Furthermore, certain vectors are capable of directing the expression of genes to which they are operably linked. Such vector is referred to herein as “expression vector.” Common expression vectors used in recombinant DNA techniques are usually in the form of plasmids.

Recombinant expression vectors may comprise a nucleic acid of the present invention in a form suitable for expression of the nucleic acid in a host cell, which means that these recombinant expression vectors comprise one or more regulatory elements selected on the basis of the host cell to be used for expression, the regulatory element is operably linked to the nucleic acid sequence to be expressed. In a recombinant expression vector, “operably linked” is intended to mean that the nucleotide sequence of interest is linked to the one or more regulatory elements in a manner that allows the expression of the nucleotide sequence (e.g., in an in vitro transcription/translation system or when the vector is introduced into the host cell, in the host cell).

The term “regulatory element” is intended to include promoter, enhancer, internal ribosomal entry site (IRES), and other expression control elements (e.g., transcription termination signal, such as polyadenylation signal and poly U sequence). Such regulatory sequences are described, for example, in Goeddel, GENE EXPRESSION TECHNOLOGY: METHODS IN ENZYMOLOGY, 185, Academic Press, San Diego, California, 1990. Regulatory elements include those sequences that direct the constitutive expression of a nucleotide sequence in many types of host cells as well as those sequences (e.g., tissue-specific regulatory sequences) that direct the expression of the nucleotide sequence only in certain host cells. A tissue-specific promoter may primarily direct expression in a desired tissue of interest, and the examples of the tissue include muscle, neuron, bone, skin, blood, specific organ (e.g., liver, pancreas), or particular cell type (e.g., lymphocyte). Regulatory elements may also direct expression in a timing-dependent manner (e.g., in a cell cycle-dependent or developmental stage-dependent manner), and the manner may or may not be tissue- or cell type-specific.

Those skilled in the art will appreciate that the design of expression vector may depend on factors such as the choice of host cell to be transformed, the level of expression desired, and the like. A vector can be introduced into a host cell to thereby produce transcript, protein, or peptide, including fusion protein or peptide encoded by the nucleic acid as described herein (e.g., clustered regularly interspaced short palindromic repeats (CRISPR) transcript, protein, enzyme, mutant form thereof, fusion protein thereof, etc.).

### Embodiments of the Present Invention

The present invention provides the following embodiments:
1. A CRISPR-based target gene imaging system, comprising:
(1) a dCas9-expressing vector;
(2) an engineered sgRNA-expressing vector, the engineered sgRNA comprising: a sgRNA backbone containing n copies of RNA aptamer, and a sgRNA sequence specific for a target gene to be detected, wherein n is an integer greater than or equal to 2; and
(3) a fusion protein-expressing vector, the fusion protein comprising: an RNA binding motif that specifically recognizes the RNA aptamer, a multimerization peptide segment and a fluorescent protein, which are operably linked.
2. The imaging system according to embodiment 1, wherein the engineered sgRNA-expressing vector is driven by a U6 promoter, preferably, the U6 promoter is a mouse U6 promoter (mU6) or a human U6 promoter (hU6).
3. The imaging system according to embodiment 1, wherein the RNA aptamer and the RNA binding motif are present in a paired combination selected from the group consisting of: PP7 and PCP, MS2 and MCP, or BoxB and N22.
4. The imaging system according to embodiment 1, wherein n is 2, 3, 4, 5, 6, 7 or 8.
5. The imaging system according to embodiment 1, wherein the n copies of RNA aptamer are linked in series, preferably through a linker.
6. The imaging system according to embodiment 1, wherein the multimerization peptide segment is foldon trimerization small peptide, GCN4 trimerization small peptide, 3HB trimerization small peptide, 6G6H hexamerization small peptide, or sDscama30 dimerization small peptide, and wherein the multimerization peptide segment is fused to the N-terminal or C-terminal of the fluorescent protein, or located at the N-terminal or C-terminal of the fusion protein, preferably, the multimerization peptide segment is located at the N-terminal of the fusion protein.
7. The imaging system according to embodiment 1, wherein the fluorescent protein is green fluorescent protein (GFP), enhanced green fluorescent protein (eGFP), red fluorescent protein (RFP), or blue fluorescent protein (BFP).
8. The imaging system according to embodiment 1, wherein the fusion protein-expressing vector further comprises a nuclear localization sequence (NLS).
9. The imaging system according to embodiment 1, wherein the dCas9-expressing vector is transfected into a cell line.
10. A CRISPR-based imaging system, comprising:
(1) a dCas9 protein;
(2) an engineered sgRNA-expressing vector, the engineered sgRNA comprising: an sgRNA backbone containing n copies of RNA aptamer, and a sgRNA sequence specific for a target gene to be detected, wherein n is an integer greater than or equal to 2;
(3) a fusion protein-expressing vector, the fusion protein comprising: an RNA binding motif that specifically recognizes the RNA aptamer, a multimerization peptide segment and a fluorescent protein, which are operably linked.
11. The imaging system according to embodiment 10, wherein the RNA aptamer and the RNA binding motif are present in a paired combination selected from the group consisting of: PP7 and PCP, MS2 and MCP, or BoxB and N22.
12. The imaging system according to embodiment 10, wherein n is 2, 3, 4, 5, 6, 7 or 8.
13. The imaging system according to embodiment 10, wherein the n copies of RNA aptamer are linked in series, preferably via a linker.
14. The imaging system according to embodiment 10, wherein the multimerization peptide segment is foldon trimerization small peptide, GCN4 trimerization small peptide, 3HB trimerization small peptide, 6G6H hexamerization small peptide, or sDscama30 dimerization small peptide, and wherein the multimerization peptide segment is fused to the N-terminal or C-terminal of the fluorescent protein, or located at the N-terminal or C-terminal of the fusion protein, preferably, the multimerization peptide segment is located at the N-terminal of the fusion protein.
15. The imaging system according to embodiment 10, wherein the fluorescent protein is green fluorescent protein (GFP), enhanced green fluorescent protein (EGFP), red fluorescent protein (RFP) or blue fluorescent protein (BFP).
16. The imaging system according to embodiment 10, wherein the fusion protein-expressing vector further comprises a nuclear localization sequence (NLS).
17. A CRISPR-based live cell target gene imaging method, the method comprising:
(i) constructing the CRISPR-based imaging system according to any one of embodiments 1-9;
(ii) transfecting a cell to be detected with each of the expression vectors in the imaging system; and
(iii) observing aggregation spots formed by the imaging system by using a confocal microscope.
18. The method according to embodiment 17, wherein the method is used for labeling and imaging a single-copy or multi-copy gene in a living cell.
19. The method according to embodiment 18, wherein the gene is a chromosomal DNA or extra-chromosomal DNA.
20. The method according to embodiment 18, wherein the gene is an extrachromatin circular DNA element (eccDNA).
21. A CRISPR-based live cell target gene imaging method, the method comprising:
(i) constructing the CRISPR-based imaging system according to any one of embodiments 10-16;
(ii) transfecting a cell to be detected with the dCas9 protein, the engineered sgRNA-expressing vector, and the fusion protein-expressing vector in the imaging system; and
(iii) observing aggregation spots formed by the imaging system by using a confocal microscope.
22. The method according to embodiment 21, wherein the cell to be detected is transfected with the dCas9 protein, the engineered sgRNA-expressing vector and the fusion protein-expressing vector in the imaging system by electroporation.
23. A kit for CRISPR-based target gene labeling and imaging, the kit comprising the dCas9-expressing vector, the engineered sgRNA-expressing vector and the fusion protein-expressing vector of the CRISPR-based imaging system according to in any one of embodiments 1-9, wherein the dCas9-expressing vector, the engineered sgRNA-expressing vector and the fusion protein-expressing vector are each stored in a separate container.
24. A kit for CRISPR-based target gene labeling and imaging, the kit comprising the dCas9 protein, the engineered sgRNA-expressing vector and the fusion protein-expressing vector of the CRISPR-based imaging system according to any one of embodiments 10-16, wherein the dCas9 protein, the engineered sgRNA-expressing vector and the fusion protein-expressing vector are each stored in a separate container.

### EXAMPLES

By referring to the following examples, those skilled in the art will be more aware of the technical solutions and technical effects of the present invention. Those skilled in the art should understand that the following examples are only for the purpose of illustration, and are not interpreted as limiting the protection scope of the present invention in any way. The protection scope of the present invention is defined by the claims. Without departing from the spirit and scope of the present invention, those skilled in the art can make corresponding modifications to the embodiments of the present invention, and these modifications are also included in the scope of the present invention.

The following Table 1 and Table 2 list the main experimental instruments and main reagents and medicines used in the following examples. Unless otherwise specified, the reagents or medicines used in the examples were all commercially available.

TABLE 1
Main experimental instruments

| Instrument Name | Cat. No./Model | Company |
|---|---|---|
| 4° C. Centrifuge | Multifuge X1R | Eppendorf/ |
| | | Beckman |
| Biological safety cabinet | 51025411 | Thermo |
| CO₂ biochemical incubator | 4111FO | Thermo |
| Electric constant temperature | DK-S12 | Shanghai |
| water bath | | Senxin |
| Micropipette | 4920000903 | Eppendorf |
| Electric constant temperature | BGZ-70 | Shanghai |
| blast drying oven | | Jinghong |
| Horizontal shaker | WD-9405B | Beijing Liuyi |
| Fluorescence Inverted Microscope | TE2000 | Nikon |
| Liquid nitrogen tank | Locator6Plus | Thermo |
| | (CY509109CN) | |
| Analytical balance | BS 124S | Sartorius |
| Horizontal gel electrophoresis | DYCP-31C | Beijing Liuyi |
| instrument | | |
| Benchtop high-speed centrifuge | 5401000099 | Eppendorf |
| Light microscope | TS2-BF-L | Olympus |
| Air constant temperature shaker | KYC 100B | Shanghai Foma |
| Clean work bench | LVG-3AG-F8 | ESCO |
| DM4B upright fluorescence | DM4B | Leica |
| microscope | | |
| Laser confocal petri dish | 801001 | Nest |
| Laser confocal microscope | Dragonfly 200 | Andor |

TABLE 2
Reagents and medicines

| Name | Cat. No. | Company |
|---|---|---|
| Ampicillin | A100339-0005 | Amresco |
| Fetal bovine serum (FBS) | 10099141C | Hyclone |
| Lipofectamine 2000 | 11668019 | Invitrogen |
| Dimethyl sulfoxide (DMSO) | D8371 | Sigma |
| DMEM high-glucose medium | C11995500CP | Hyclone |
| Opti-Medium | 31985070 | Gibco |
| Trypsin | 25200072 | Hyclone |
| PBS | SH30256.01 | Hyclone |
| DH5α | C502-03 | Vazyme |

### Example 1. Construction of CRISPR FISHer System

The constructed CRISPR FISHer system comprised:
(1) U2OS cell line stably expressing dCas9: Firstly, dCas9 expression element was constructed into a lentiviral packaging system, and then the system was transfected into 293T cell line to obtain a viral supernatant. Finally, the wild-type U2OS cell line was infected with the virus supernatant, and the U2OS cell line stably expressing dCas9 was obtained by screening;
(2) mU6-sgRNA-2×/8×PP7-expressing vector: this vector expressed sgRNA, the sgRNA recognized a genome to be detected, guided dCas9 to bind thereto, and a stable 2×PP7 element or 8×PP7 element was inserted into the sgRNA backbone. Wherein mU6 was a promoter for sgRNA, and its nucleotide sequence was set forth in SEQ ID No: 8.

PP7 was present in a binding region of other RNA binding motifs except Cas9 on the guide RNA (sgRNA), and generally bound to PCP. PP7 existed in a stem-loop structure. Several kinds of PP7 commonly used in this field are as follows:

GGAGCAGACGATATGGCGTCGCTCC
CCAGCAGAGCATATGGGCTCGCTGG
CGAGCAGACCATATGGGGTCGCTCG
GAAGCAGAAGATATGGCTTCGCTTC
CAAGCAGAACATATGGGTTCGCTTG
(3) Foldon-GFP-PCP-expressing vector
The amino acid sequence of the constructed
Foldon-GFP-PCP element is set forth in SEQ ID
No: 22.

In application, firstly, the expressed Foldon-GFP-PCP fusion protein could spontaneously form a protein trimer, and secondly, PCP could specifically bind to PP7, that was, the Foldon-GFP-PCP fusion protein would bind to the PP7 element in the sgRNA.

Specifically, the aggregation process was as follows:

The sgRNA first bound to the dCas9 protein to form a complex, then dCas9/sgRNA bound to a DNA sequence of a sgRNA target, and then PP7 at the stem-loop on the sgRNA could recruit the trimerized Foldon-GFP-PCP fusion protein (as shown in FIG. 5A).

Since the trimerized Foldon-GFP-PCP fusion protein had three PCP domains, it could also bind to PP7 at the stem-loop of other sgRNAs in addition to the stem-loop PP7 that formed the complex of dCas9 protein and sgRNA. The other sgRNAs also recruited more trimerized Foldon-GFP-PCP fusion proteins. Therefore, the system of the present invention would eventually form an aggregate of sgRNA-PP7-Foldon-GFP-PCP through repeated recruitment and binding of sgRNA and trimerized PCP-Foldon-GFP fusion protein. This aggregate would contain multiple GFP fluorophores, thereby achieving n-fold amplification of fluorescence signal (n is greater than or equal to 3 folds of the number of PCP stem-loop in the sgRNA) (FIG. 8B).
(3) Multiple sgRNAs and green fluorescent proteins (GFP) would aggregate around the target sequence, which greatly increased the resolution and signal/background ratio of the system, and finally achieved the effect of successful labeling and imaging of a single-copy gene by using only one sgRNA.

### Plasmid Transformation and Extraction:

Referring to the method of Ma Hanhui [11], the constructed dCas9-expressing vector, mU6-sgRNA-8PP7-expressing vector and PCP-foldon-GFP-expressing vector were transformed into E. coli DH5a cells, and the plasmids were amplified. The high-purity plasmid mini-extraction kit (DP104) of Tiangen Biochemical Technology (Beijing) Co., Ltd. was used to extract various plasmids.

### Cell Culture and Subculture:

Referring to the method of Ma Hanhui [11], the cell culture and passage were performed.

Lipofectamine 2000 plasmid transient transfection:
(1) Cells were cultured overnight, and the cell density should reach 40-50% by the time of transfection;
(2) Plasmid was diluted in Opti-MEM (its amount was selected according to Table 3), vortexed, and then allowed to stand for 5 minutes;
(3) Lipofectamine 2000 was diluted in Opti-MEM (its amount was selected according to Table 3), vortexed, and then allowed to stand for 5 minutes;

TABLE 3
Plasmid transfection system shown as follows:

| Size of dish/plate | Transfection medium | Plasmid | Liposome | DMEM |
|---|---|---|---|---|
| 3.5 cm dish | 2 × 100 μL | 4.0 μg | 10 μL | 2 mL |

(4) The pre-diluted liposome (included in the Lipofectamine 2000 kit (purchased from Invitrogen)) and the plasmid were mixed well, vortexed, and then allowed to stand for 20 min;
(5) The mixed solution after standing was slowly added dropwise into a petri dish, and the petri dish was shaken gently for mixing well;
(6) Culture was then performed in a cell culture incubator at 37° C. for 12 h;
(7) After 12 hours, the cell state was observed, the cell culture medium was replaced, and pictures were taken with a fluorescence microscope.

### Preparation of Protein Samples:

Protein samples were prepared according to conventional methods in the art.

### Determination of Protein Concentration by BCA Method:

Referring to the method of Ma Hanhui [11], the BCA method was used to determine protein concentrations.

### Example 2. Verification of Foldon-GFP Trimerization

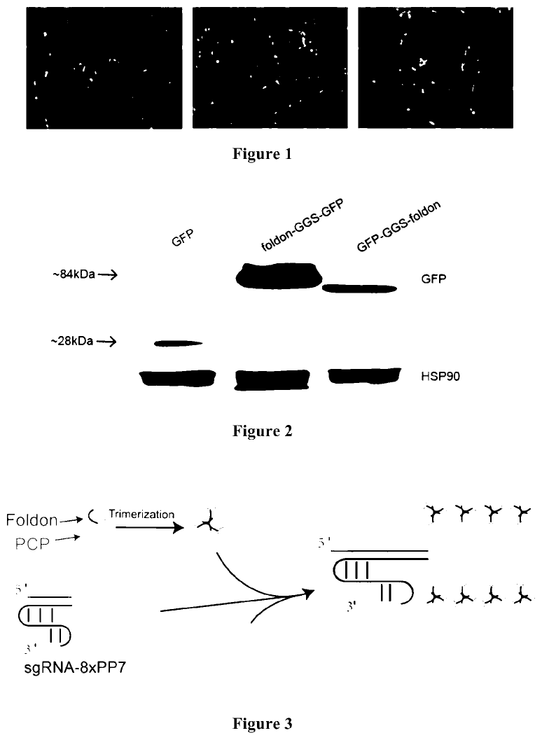

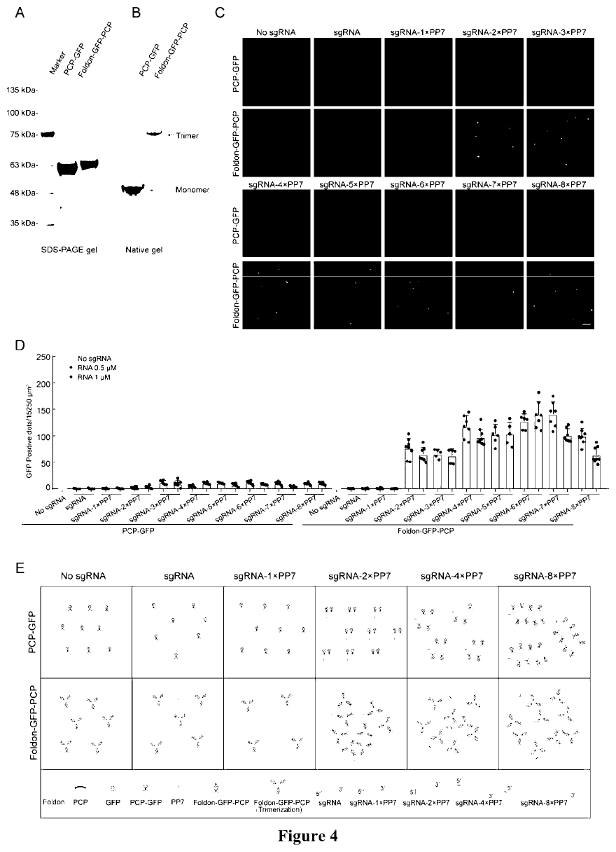

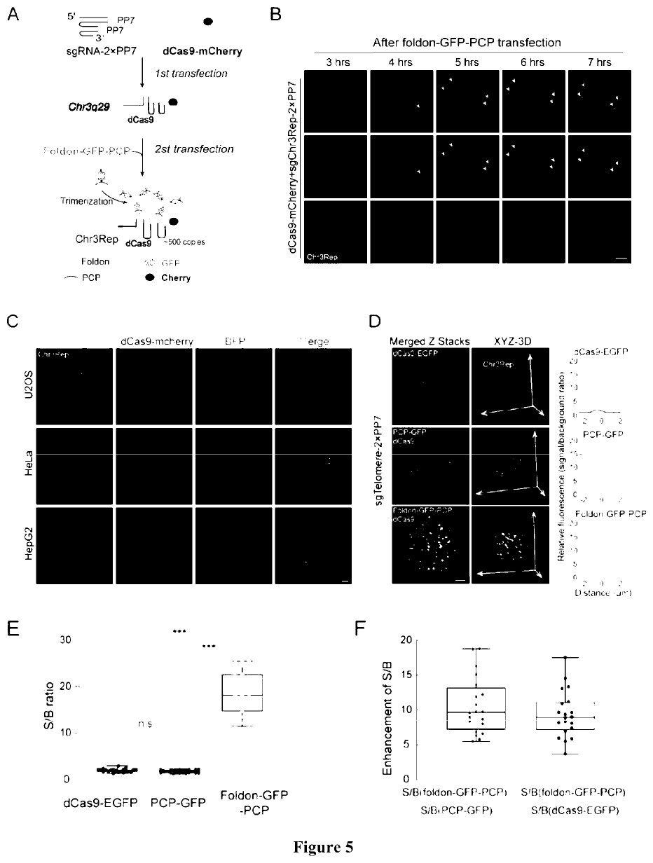

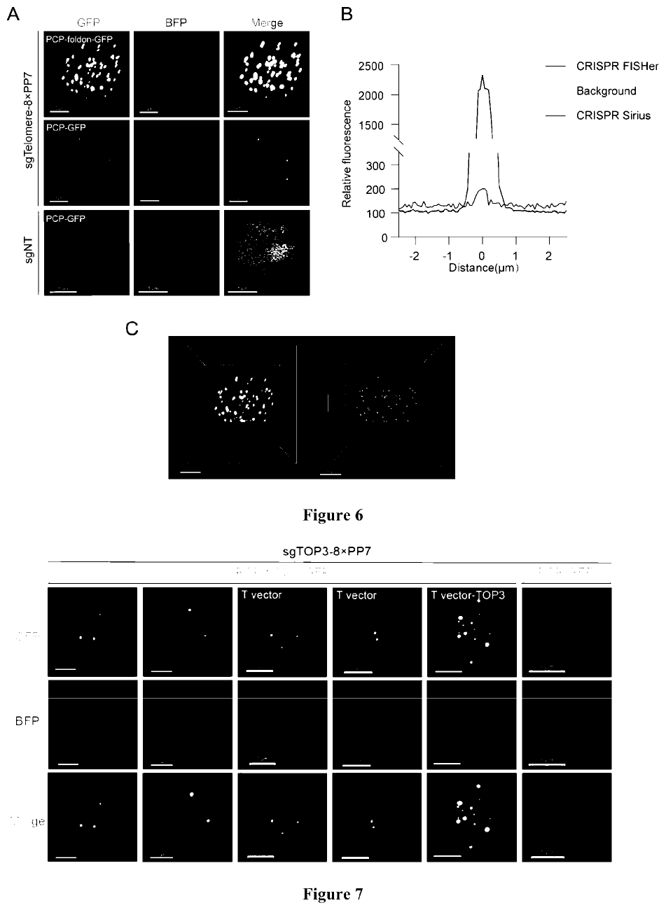

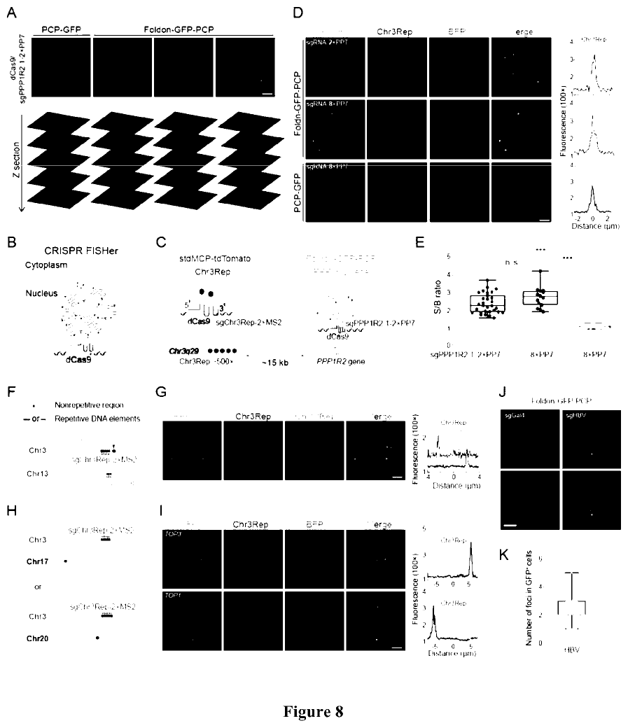

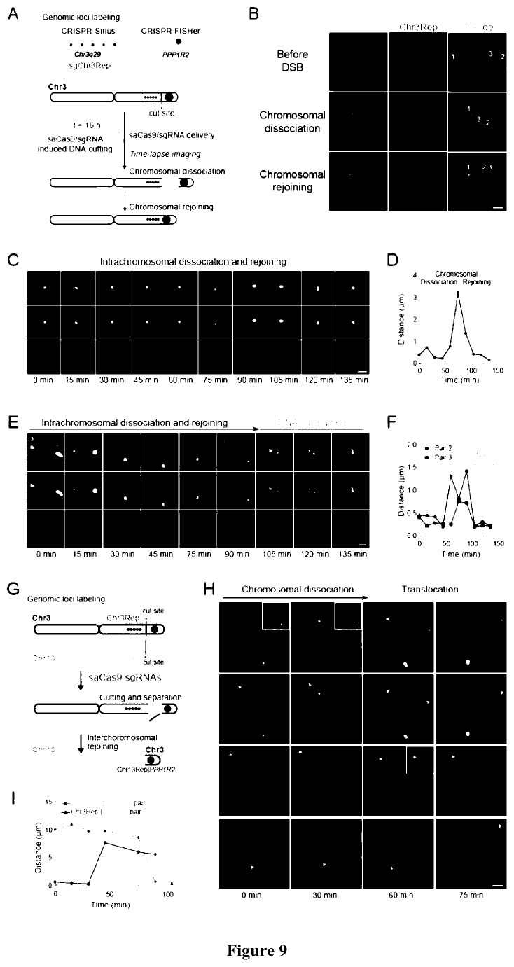

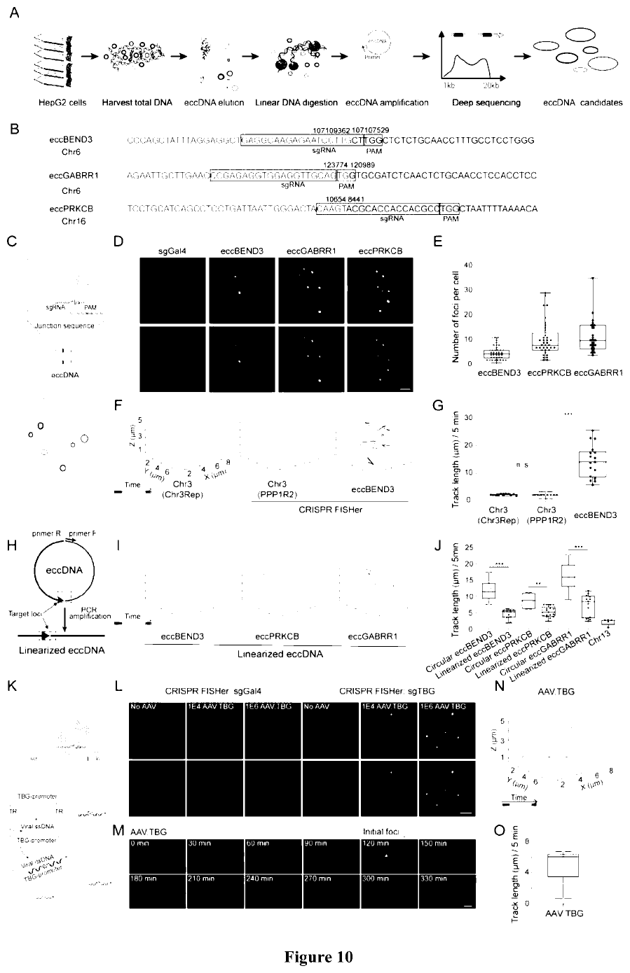

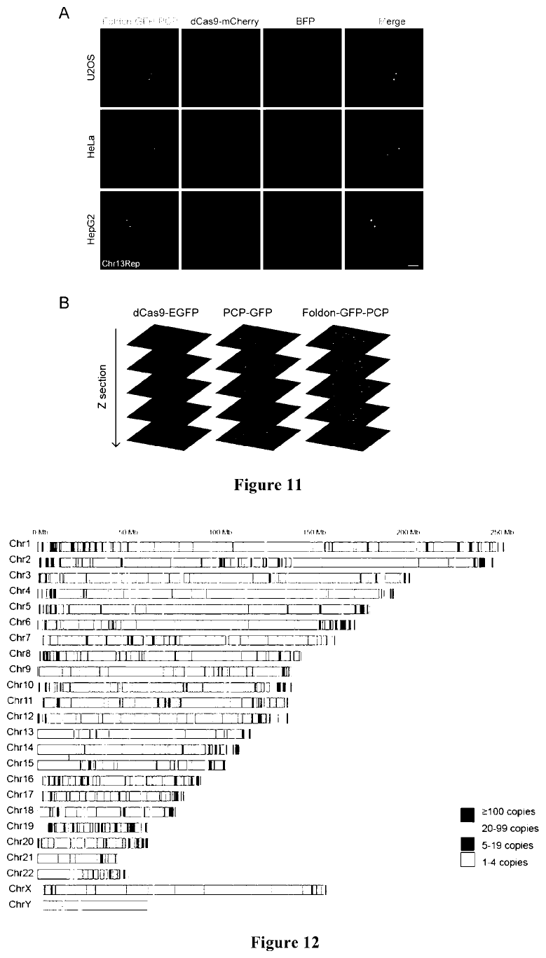

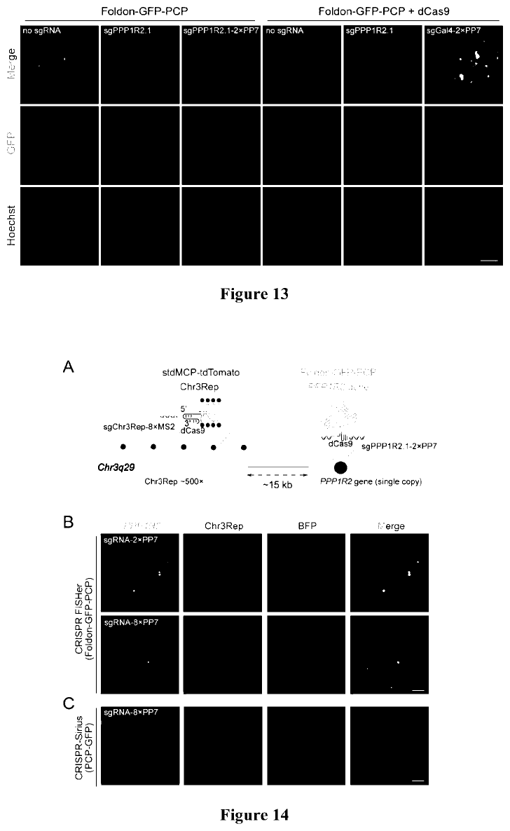

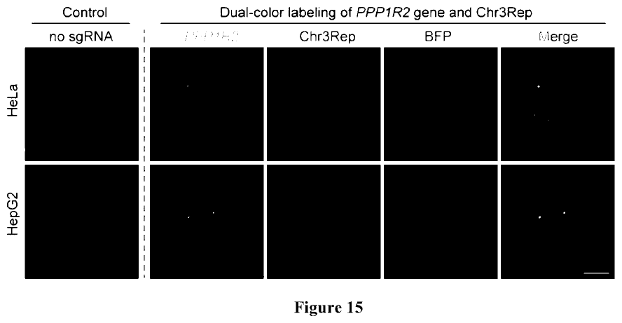

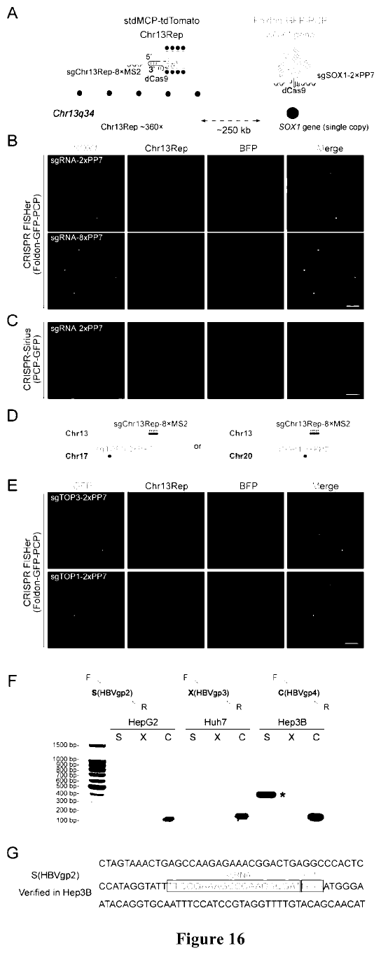

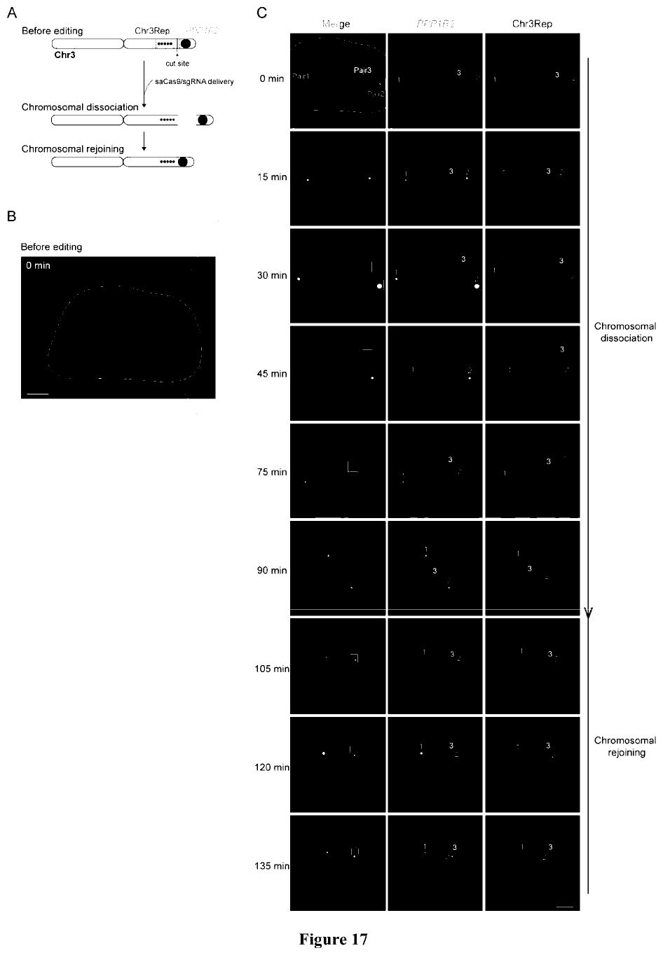

According to the standard molecular cloning method, foldon element was fused with GFP (foldon was fused to the N-terminal or C-terminal of GFP). A fusion protein-expressing vector was constructed, and then transfected into 293T cells. The cells were harvested 12 hours after transfection, the protein was extracted, Western blot (western blot) native gel was used to detect the GFP trimerization, the results were shown in FIG. 1 and FIG. 2.

FIG. 1 shows the fluorescence of fusion construct of the foldon element and GFP expressed in 293T cells for 12 hours. It can be seen that whether in the control group (left column, only GFP) or the experimental groups (middle column and right column, foldon was fused to the N-terminal or C-terminal of GFP, respectively), after transfection of 12 hours, the fluorescence intensity had reached a near-saturation state. FIG. 2 shows the western blot native gel detection results of GFP. Wherein, GGS schematically represented a linker sequence. It can be seen that compared with the GFP of the control group (wild type, left lane), the trimerization of GFP occurred no matter whether the foldon element was fused at the N-terminal (middle lane) or the C-terminal (right lane) of GFP, but the trimerization effect of the fusion of foldon at the N-terminal of GFP was stronger than that of the fusion at the C-terminal.

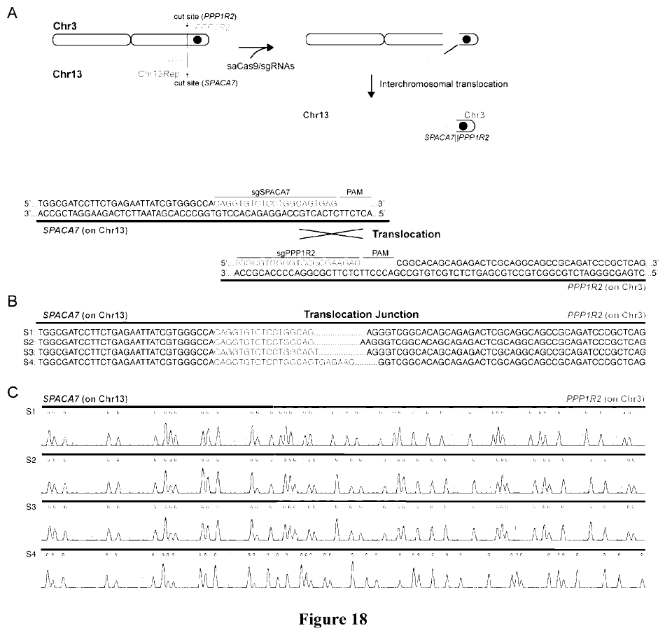

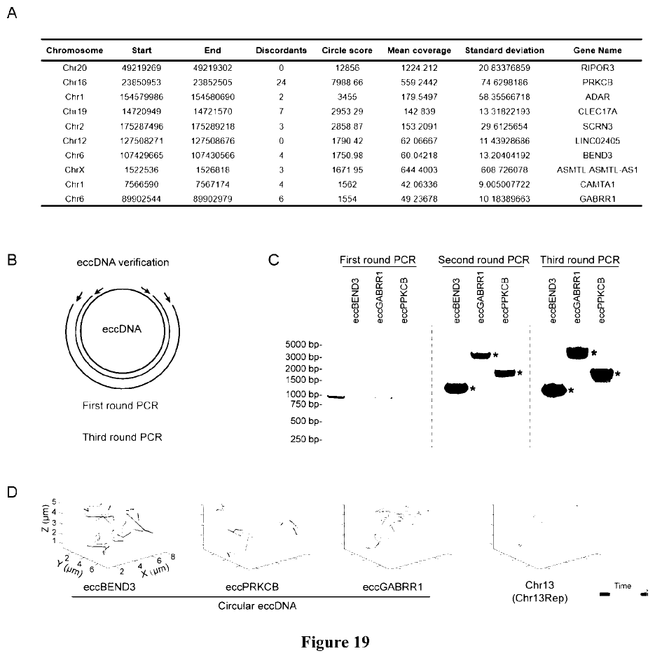

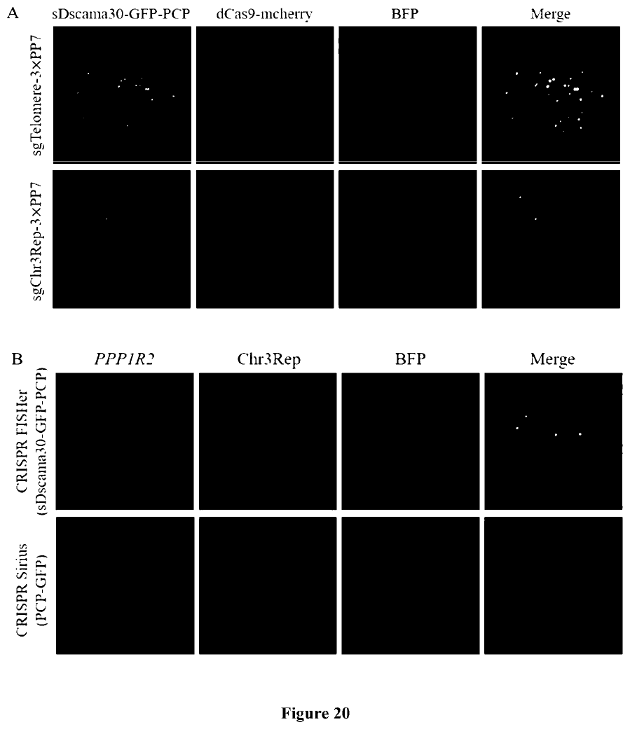

FIG. 4 shows the bands separated by electrophoresis under denaturing (A, SDS-PAGE gel) and non-denaturing (B, non-denaturing gel) conditions of purified foldon-GFP-PCP and PCP-GFP fusion proteins. It can be seen that the foldon-GFP-PCP could undergo trimerization compared with PCP-GFP in the control group (FIG. 4B).

The results in FIG. 2 and FIG. 4 demonstrate that the fusion of the foldon element to a target protein (e.g., a fluorescent protein, for example, but not limited to, GFP) would promote the trimerization of the target protein.

### Example 3. Using PCP-Foldon-GFP-Based CRISPR FISHer System to Label and Image Telomeres

In order to label-and-image telomeres in living cells, the sgRNA part of the mU6-sgRNA-8×PP7-expressing vector prepared in Example 1 was made to be telomere-specific (which could be expressed as mU6-sgTelomere-8×PP7-expressing vector, shown d as “sgTel-8PP7” in Table 4, wherein “sgTelomere” or “sgTel” indicated sgRNA targeting to telomere). 293T cells were co-transfected with dCas9-expressing vector (e.g., CMV-dCas9), mU6-sgTelomere-8×PP7-expressing vector and PCP-foldon-GFP-expressing vector, the cells were harvested 12 hours after transfection, and the fluorescence expression was detected with laser confocal microscope.

TABLE 4
Co-transfection system

| First group | Second group | Third group | Trans- |
|---|---|---|---|
| (experimental | (control | (control | fection |
| group) | group) | group) | amount |
| sgTel-8PP7 | sgTel-8PP7 | sgNT-8PP7 | 200 ng |
| CMV-dCas9 | CMV-dCas9 | CMV-dCas9 | 500 ng |
| EFS-PCP-foldon-GFPnls | EFS-PCP-GFPnls | EFS-PCP-GFPnls | 20 ng |

CMV indicates a promoter for dCas9, EFS indicates a promoter for PCP-GFP, and nls indicates a nuclear localization sequence.

The results of fluorescence imaging and fluorescence intensity analysis were shown in FIG. 6 (A and B). The results show that, compared with the CRISPR-Sirius imaging results of the control group (FIG. 6B, the blue curve, that was, the curve showing the secondary peak), the intensity of the fluorescent dots of the CRISPR FISHer system of the present invention was stronger, and the resolution and signal/background ratio both had a very obvious improvement, and there was almost no background signal (FIG. 6B, red curve, that was, the curve showing the highest peak).

FIG. 6C shows the 3D imaging results of the cells in the experimental group. At the same time, the imaris software was used to count the fluorescence labeling points in the cells at a threshold of 0.2 μm. As a result, there were 94 green fluorescent dots, which was very close to the number of telomeres in 293T cells (92). This result showed that the accuracy of the CRISPR FISHer system of the present invention in labeling genome loci was also very high.

### Example 4. Using Foldon-GFP-PCP-Based CRISPR FISHer System to Label and Image Telomeres

The sgRNA part of the mU6-sgRNA-2×PP7-expressing vector prepared in Example 1 was made to be telomere-specific (which could be expressed as mU6-sgTelomere-2×PP7-expressing vector, and shown as “sgTel-2PP7” in Table 5). U2OS cells were co-transfected with dCas9-expressing vector (e.g., CMV-dCas9), mU6-sgTelomere-2×PP7-expressing vector and Foldon-GFP-PCP-expressing vector, and dCas9-EGFP and PCP-GFP were used as controls. The cells were harvested 16 hours after transfection, and the fluorescence expression was detected by confocal laser microscopy.

TABLE 5
Co-transfection system

| First group | Second group | Third group | Trans- |
|---|---|---|---|
| (experimental | (control | (control | fection |
| group) | group) | group) | amount |
| sgTel | sgTel-2PP7 | sgTel-2PP7 | 1000 ng |
| dCas9-EGFP | CMV-dCas9 | CMV-dCas9 | 1000 ng |
| | EFS-PCP-GFPnls | Foldon-GFP-PCP | 2000 ng |

The results of fluorescence imaging and fluorescence intensity analysis were shown in FIG. 5 (D-F). FIG. 5D showed the GFP fluorescence imaging results of labeled telomeres in the experimental group (with foldon) and the control groups (without foldon) under the same transfection conditions, and FIGS. 5E and 5F showed the comparison of signal/background ratio for these three groups. Among them, 2×PP7 was inserted into the sgRNA targeting telomeres (sgTelomere-2×PP7), the experimental group expressed dCas9, sgTelomere-2×PP7 and foldon-PCP-GFP; the control group 1 expressed dCas9-EGFP and sgTelomere-2×PP7; and the control group 2 expressed dCas9, sgTelomere-2×PP7 and PCP-GFP (no foldon). In this version of the CRISPR FISHer system, the signal/background ratio of the experimental group could reach up to 10 times that of the control group.

At the same time, in order to explore whether foldon-GFP-PCP could aggregate at a target site, we first used a repetitive genome region of Chr3q29 (about 500 repeats, named Chr3Rep) as a labeling object, used dCas9-mCherry and sgRNA-2×PP7 to target Chr3Rep, and then expressed Foldon-GFP-PCP plasmid in human osteosarcoma cells U2OS (FIG. 5A). According to the results of fluorescence imaging, foldon-GFP-PCP appeared as early as 4 hours after transfection into the nucleus, co-localized with the Chr3Rep site, and gradually became brighter and clearer (FIG. 5B). These results suggest that target DNA-bound dCas9/sgChr3Rep potentially recruited foldon-GFP-PCP to the target site while enhancing the GFP signal at the target site and reducing nonspecific background. Simultaneously, in HeLa and HepG2 cells, the co-localization was further analyzed. As expected, 24 hours after transfection, Foldon-GFP-PCP co-localized well with dCas9-mCherry (FIG. 5C). To further examine the specificity of dCas9/sgRNA-2×PP7-induced foldon-GFP-PCP localization at target site, we utilized another sgRNA targeting to Chr13q34 repeat element (about 350 repeats, referred as Chr13Rep), and this specific signal was verified (FIG. 11A).

### Example 5. Using PCP-Foldon-GFP-Based CRISPR FISHer System to Label and Image Single-Copy Gene TOP3

TOP3 gene is a single-copy gene encoding human DNA topoisomerase III, located on p11.2-12 of human chromosome 17 [23].

For imaging and labeling single-copy gene TOP3, three plasmids were constructed as described in Example 1: dCas9-expressing vector (e.g., CMV-dCas9), sgTOP3-8×PP7-expressing vector (i.e., the sgRNA part in mU6-sgRNA-8×PP7-expressing vector was made TOP3-specific) and PCP-foldon-GFP-expressing vector. These three expression vectors were co-transfected into 293T cells, the cells were harvested after 12 hours, and their fluorescence expression was detected by laser confocal microscope.

TABLE 6
Co-transfection system

| | | | | Trans- |
|---|---|---|---|---|
| First group/ | | Third group/ | | fection |
| second group | Fifth group | forth group | Sixth group | amount |
| sgTOP3-8PP7 | sgTOP3-8PP7 | sgNT-8PP7 | sgTOP3-8PP7 | 200 ng |
| CMV-dCas9 | CMV-dCas9 | CMV-dCas9 | CMV-dCas9 | 500 ng |
| EFS-PCP- | EFS-PCP- | EFS-PCP- | EFS-PCP- | 200 ng |
| foldon- | foldon- | foldon- | GFPnls | |
| GFPnls | GFPnls | GFPnls | | |
| | T-vector TOP3 | T vector | | 200 ng |

T-vector represents a T vector;
T-vector TOP3 represents a T vector containing a sequence of TOP3 gene

The results of GFP fluorescence detection were shown in FIG. 7. FIG. 7 showed the fluorescence detection results of the experimental groups (the first two columns from the left) and the control group in labeling single copy TOP3 gene under the same transfection conditions:
(1) The results of the first group and the second group (the first to second columns from the left) were all labeling results of TOP 3 gene, in which two fluorescence dots and four fluorescence dots represented the positions of the gene before and after replication, respectively;
(2) In the fifth group (the fifth column from the left), a sequence from the TOP 3 gene was exogenously transfected through “T-vector TOP3” on the basis of the experiments of the first group and the second group, the sequence was the sgRNA targeting sequence of the first group and the second group, and the results showed that the number of fluorescence dots increased significantly;
(3) In the third and fourth groups (the third to forth columns from the left), a backbone of T vector (that was, without TOP 3 gene sequence) was exogenously transfected on the basis of the experiments of the first and second groups. The results were similar to the first group and the second group, which indicated that the introduction of the backbone of T vector in the experiment of the third group had no effect on the experimental results;
(4) The sixth group (the sixth column from the left) was a control experiment using the CRISPR Sirius system. The fusion protein was PCP-GFP (that was, without foldon). The results show that the green fluorescence was diffusely distributed, and the corresponding single copy loci could not be accurately labeled.

The results of Example 5 (shown in FIG. 7) showed that the CRISPR FISHer system of the present invention could very sensitively and accurately label single-copy genes, and the fluorescence intensity and signal/background ratio had been significantly improved. Therefore, the CRISPR FISHer system of the present invention can well solve the current problems of “difficult to achieve non-repetitive gene labeling” and “low signal/background ratio” in the field of CRISPR imaging. It provides a good indicator tool for a deeper understanding of gene dynamic changes such as gene transcription and translation.

### Example 6. Foldon-GFP-PCP-Based CRISPR FISHer Realizes Live Cell Imaging of Non-Repetitive Region in Chromosomal DNA or Extra Chromosomal DNA

Non-repetitive genome regions comprise about 65% of the human genome and include almost all protein-coding genes (FIG. 12). Therefore, first we applied the CRISPR FISHer system to target non-repetitive genome regions in living cells. We established a U2OS cell line stably expressing dCas9. The sgRNA (sgPPP1R2) targeted to a single-copy gene, PPPIR2, located at Chr3q29, and had a distance about 36 kb from the Chr3q29 repetitive region. We co-transfected U2OS-dCas9 cells with plasmids expressing PCP-GFP or foldon-GFP-PCP and sgPPP1R2-2×PP7. Different from the diffuse green signal of PCP-GFP and sgPPP1R2-2×PP7 groups, we observed bright GFP-labeling fluorescence signal dots in the cells expressing foldon-GFP-PCP and sgPPP1R2-2×PP7, which indicated that we could image single-copy gene PPP1R2 at Chr3q29 by using CRISPR FISHer (FIGS. 8A to 8C). Furthermore, in the control cells without dCas9 or transfected with wild-type sgRNA or transfected with sgGal4 (not targeting human genome DNA), we observed that the green signal diffused throughout the cell nucleus or aggregated in the nucleolus (FIG. 13).

In order to verify the specificity to the non-repetitive DNA region labeled by CRISPR FISHer, we used CRISPR FISHer to label PPPIR2 gene, and used 2×MS2 or 8×MS2 CRISPR system as an internal reference to label Chr3Rep (FIG. 8C and FIG. 14A). As expected, the two sites of CRISPR FISHer targeting to sgRNA-2×PP7 or sgRNA-8×PP7 were highly co-localized in most U2OS cells as well as HeLa and HepG2 cells (FIGS. 8D to 8E, FIG. 15). At the same time, we made statistics on the signal/background ratios of the CRISPR FISHer system and the CRISPR-Sirius in labeling PPP IR2 gene in different U2OS cells. We found that, compared to the CRISPR-Sirius system with diffuse green signal, the CRISPR FISHer system could clearly label the single-copy gene with a signal/background ratio of up to 4 (FIG. 8E).

Next, to further test the specificity of CRISPR FISHer in labeling non-repetitive regions, we implemented three additional different strategies. First, we utilized another single-copy gene, SOX1 (about 250 kb Chr13Rep Chr13) (FIG. 16A), and found that the CRISPR FISHer-labeled SOX1 gene locus nearly coincided with the Chr13Rep locus (FIGS. 16B to 16C). Second, we labeled Chr3Rep and Chr13Rep with different fluorescent proteins and found that sgPPPIR2-2×PP7 co-localized with sgChr3Rep-tdTomato, but not with sgChr13Rep-Halo (FIGS. 8F to 8G). Finally, we collectively imaged and labeled Chr3Rep, TOP3 on Chr17 and TOPI on Chr20 in U2OS cells (FIG. 8G). We found that the CRISPR FISHer signals of TOP3 and TOPI did not co-localize with the signal of Chr3Rep (FIG. 81), nor with Chr13Rep (FIG. 16D to 16E)

Furthermore, we extended the application of CRISPR FISHer to Hep3B cells to detect hepatitis B virus (HBV). We found that, compared with the diffuse green fluorescence signal of sgGal4 in the control group, the sgRNA targeting HBV could present a clear green dot-like signal (FIGS. 8J to 8K, FIGS. 16F to 16G).

### Example 7. Using CRISPR FISHer System to Track CRISPR-Induced Double-Strand Breakage and Non-Homologous End-Joining Repair

CRISPR-induced double-strand breakage (DSB) is mostly repaired by non-homologous end-joining (NHEJ), and NHEJ has been applied in gene therapy to silence single or multiple targeted genes. We extended the application of CRISPR FISHer to track the real-time dynamics of CRISPR-Cas9-induced DSB and subsequent NHEJ repair process in living cells. To achieve genome DNA locus imaging and DSB induction in the same cell, we introduced SaCas9/sgRNA to mediate DNA cleavage in addition to SpCas9-based genome labeling. We first delivered a SpCas9-based imaging system in U2OS cells so as to use the CRISPR FISHer system (sgPPPIR2-2×PP7-GFP) to label single-copy gene PPP 1R2 and to use the CRISPR Sirus (sgChr3Rep-8×MS2-tdTomato) to label the repetitive Chr3q29 region; 12 hours later, we electrotransferred the SaCas9/sgRNA system targeting PPPIR2 gene (SaCas9/sgPPP1R2.2) onto Chr3 to induce DSB generated between the gene loci labeled with sgPPP1R2.2-2×PP7 and sgChr3Rep (FIG. 9A). Sequential delivery of two orthogonal CRISPR-Cas9 systems for imaging and editing, respectively, enabled us to track DNA cleavage and repair processes at individual loci over time (FIGS. 9B to 9F, FIG. 17). For example, we captured the separation and fusion of PPP1R2 locus (green) and Chr3Rep locus (red), which might represent the entire process of SaCas9-induced DSB and NHEJ-mediated repair (FIG. 9C, FIG. 17). Remarkably, the successful DNA repair process mediated by NHEJ lasted only one hour in a single living cell (FIGS. 9B to 9C).

CRISPR-induced multiple gene editing on different chromosomes can lead to chromosomal translocation [24]. To capture the dynamics of interchromosomal rearrangements, we collectively used a SpCas9-dependent real-time imaging system (the system labeled the loci of PPPIR2 gene (sgPPP1R2.2-2×PP7) on Chr13Rep, Chr3Rep and Chr3) and a SaCas9 system (to mediate the genome cleavage between the sgPPPP1R2.2 on Chr3 and the Chr3Rep locus (SaCas9/sgPPPIR2), and the genome cleavage in the SPACA7 gene 82 kb apart from the Chr13Rep on Chr13) (FIG. 9G). After sequential delivery of the CRISPR imaging system and the CRISPR editing system, we were able to observe multiple pairs of loci targeted by sgPPP1R2.2/Chr3 and sgChr13Rep, whose distances appeared to be nearly constant (FIG. 9H), which indicated that the sgPPPIR2.2-2×PP7-labeled PPPIR2 gene on Chr3 had been successfully linked to the SPACA7 gene close to Chr13Rep. We tracked the dynamics of chromosomal translocations. Initially, the PPPIR2 and Chr13Rep loci were segregated, then moved closer, and remained together for a period of time, which might indicate the NHEJ-mediated interchromosomal repair. Finally, we verified the chromosomal translocation events by targeted sequencing (FIG. 18).

### Example 8. Using CRISPR FISHer System to Label Extrachromatin Circular DNA Element (eccDNA)

In addition to genomic DNA, extrachromatin circular DNA element (eccDNA) has been discovered for decades. It has recently been reported to function as a potent innate immune stimulator [25], whereas the visualization of specific and endogenous eccDNAs in living cells remains challenging. To target specific eccDNA, first, we isolated eccDNAs from HepG2 cells and performed next-generation sequencing (FIG. 10A). Wherein, the sequences of eccBEND3, eccGABRRI and eccPRKCB were independently verified by three rounds of PCR, TA cloning and Sanger sequencing, respectively (FIGS. 19A to 19C). The eccDNA linker sequences were chosen as targets for the CRISPR FISHer (FIG. 10B) because they were unique and did not exist in the human genome, thus enabling the CRISPR FISHer to perform specific targeting (FIG. 10C). We observed the three-dimensional distribution of CRISPR FISHer-targeted loci in HepG2 cells (FIG. 10D) and counted the number of each kind of eccDNA (FIG. 10E).

Next, we tracked the spatiotemporal dynamic movement of eccBEND3 and Chr3 targeting loci during a 5 min period (FIG. 10F), and we found that the average moving distance and space of eccBEND3 exceeded those of Chr3 (FIG. 10G), which indicated that eccDNA was highly dynamic, had longer trajectory, and moved faster. We further confirmed these dynamic differences by tracking the real-time movement of two other eccDNAs and Chr13 (FIG. 19D). Furthermore, we amplified the linear eccDNAs of eccBEND3, eccGABRRI and eccPRKCB (FIG. 10H) and tracked their dynamics (FIG. 10I). We found that the intrinsic circular eccDNA moved faster than the linear eccDNA, suggesting that this kind of circular structure was essential for the rapid movement of eccDNAs (FIG. 10J).

Herein, we develop a convenient, robust, and cost-effective CRISPR FISHer technique that enables real-time imaging of endogenous non-repetitive sequences in living cell genome or extrachromosomal DNA. To the best of our knowledge, the CRISPR FISHer strategy uses a single sgRNA to rapidly obtain native non-repetitive DNA regions in living cells with high sensitivity. The combination of sgRNA with aptamer and RNA binding protein fusion fluorescent protein and foldon peptide amplifies the local fluorescence signal. Combined with an orthogonal dCas9 imaging system, the imaging range of targeted DNA will be extended to almost all CRISPR-targeted DNA regions of interest. The CRISPR FISHer enables dynamic visualization of chromosome movement events such as DNA damage and chromosomal translocations in living cells. The visualization of extrachromatin DNA will allow us to study the function of special eccDNA from a spatiotemporal perspective. It has great potential to track multiple genomes by applying multiple orthogonal RNA aptamers in the CRISPR FISHer method. The CRISPR FISHer can be combined with other technologies such as chromosome conformation capture (3C) and Hi-C sequencing to deepen our understanding of natural chromatin spatial and dynamic organization and reveal mechanisms underlying genome higher-order structural dynamics in living cells.

### Example 9. Using CRISPR FISHer System to Label Extrachromatin Adeno-Associated Virus (AAV)

We also successfully imaged foreign-invading DNA in real time by using the CRISPR FISHer technology. Adeno-associated virus (AAV) is a non-pathogenic parvovirus that has broad application prospects in human gene therapy [26]. Double-stranded AAV DNA is generated by replication of AAV single-stranded DNA, so we can use the CRISPR FISHer system to perform targeted imaging and labeling (FIG. 10K).

For this experiment, the CRISPR FISHer system we constructed contained: a dCas9-expressing vector, a sgTBG-2×PP7-expressing vector targeting the TBG gene in the AAV genome, and a foldon-GFP-PCP-expressing vector.

First, we transfected the constructed CRISPR FISHer system into U2OS cells through 4D-nucleofector. After 12 hours, the CRISPR FISHer GFP signal was expressed and diffused in the nucleus. At this time, we added AAV particles to infect the U2OS cells. After about 120 min, both AAV and sgTBG plasmids could be observed as specific GFP fluorescent labeling signal dots in the cells, and as time went by, the green fluorescence signal gradually increased, but in the control group without AAV infection and sgGal4 plasmid transfection, we only observed a diffuse green fluorescence signal (FIGS. 10L to 10M). This demonstrated that the CRISPR FISHer system of the present invention was capable of labeling and imaging the ds AAV DNA in living cells. Remarkably, we observed the appearance of ds AAV DNA after AAV infection (FIG. 10M), suggesting that the CRISPR FISHer system of the present invention could be used to assess the number of AAV DNA molecules in living cells. Finally, we tracked the spatiotemporal movement of AAV DNA loci during a 5 min period and found that AAV single loci had high motility compared to eccDNA, but their movement was confined to a specific space, which might benefit its own transcription (FIGS. 10N to 100).

### Example 10. SDscama30-GFP-PCP Based CRISPR FISHer System can Label the Repetitive Genomic Loci by Assembling Engineered sgRNA

we co-transfected plasmids, including the plasmids for expressing sDscama30-GFP-PCP, dCas9 and sgTelomere-3×PP7/sgChr3Rep-3×PP7 into U2OS cells for repetitive genomic loci labeling and colocalization analysis. As expected, sDscama30-GFP-PCP colocalized well with dCas9-mCherry 16 hours after transfection (FIG. 20A).

TABLE 7
Co-transfection system

| | | Plasmid |
|---|---|---|
| First group | Second group | dosage |
| dCas9-mCherry | dCas9-mCherry | 500 ng |
| sgTelomere-3 × PP7 | sgChr3Rep-3 × PP7 | 500 ng |
| sDscama30-GFP-PCP | sDscama30-GFP-PCP | 500 ng |

### Example 11. With a Single sgRNA, sDscama30-GFP-PCP Based CRISPR FISHer Accomplishes the Visualization of the Endogenous Nonrepetitive Genomic Region

We wanted to use CRISPR FISHer to image the PPPIR2 gene locus in nonrepeating genomic regions in live cells. The sgRNA targeting the PPPIR2 gene (sgPPP1R2.1-7×PP7) was ˜15 kb from Chr3Rep. We transfected the plasmids into dCas9-U2OS cells to express sgPPP1R2.1-7×PP7, sDscama30-GFP-PCP, sgChr3-2×MS2 and MCP-tdTomato or sgPPP1R2.1-7×PP7, PCP-GFP, sgChr3-2×MS2, and MCP-tdTomato. We observed two bright GFP puncta for sDscama30-GFP-PCP; at the same time, the GFP signal was colocalized with the internal reference tdTomato signal of Chr3Rep, but this was not observed in control set with PCP-GFP (FIG. 20B), suggesting the capability of CRISPR FISHer to image PPPIR2 gene loci and monitor the gene copy number in U2OS cell.

TABLE 8
Co-transfection system

| | | Plasmid |
|---|---|---|
| First group | Second group | dosage |
| sgPPP1R2.1-7 × PP7 | sgPPP1R2.1-7 × PP7 | 500 ng |
| sDscama30-GFP-PCP | PCP-GFP | 500 ng |
| MCP-tdTomato | MCP-tdTomato | 500 ng |
| sgChr3Rep | sgChr3Rep-2 × MS2 | 500 ng |

Those skilled in the art will further appreciate that the present invention may be embodied in other specific forms without departing from its spirit or central characteristics. Since the foregoing description of the present invention disclosed only exemplary embodiments thereof, it should be understood that other variations are considered to be within the scope of the present invention. Therefore, the present invention is not to be limited to the particular embodiments described in detail herein. Instead, reference should be made to the appended claims as indicating the scope and content of the present invention.

### REFERENCES

1. Sawada, H. and G. F. Saunders, Transcription of Nonrepetitive DNA in Human Tissues. 1974. 34 (3): p. 516-520.
2. Langersafer, P., M. Levine, and D. C. Ward, Immunological method for mapping genes on Drosophila polytene. 1982.
3. Schwarzacher, T. and J. S. J. M. i. M. B. Heslop-Harrison, Direct fluorochrome-labeled DNA probes for direct fluorescent in situ hybridization to chromosomes. 1994. 28: p. 167.
4. Karen, D., and T. J. C. i. L. Medicine, Fluorescence In Situ Hybridization. 2011.
5. Qi, L. S., et al., Repurposing CRISPR as an RNA-guided platform for sequence-specific control of gene expression. 2013. 152 (5): p. 1173-1183.
6. Chen, B., et al., Dynamic Imaging of Genomic Loci in Living Human Cells by an Optimized CRISPR/Cas System. 2013. 155 (7): p. 1479-1491.
7. Duan, J., et al., Live imaging and tracking of genome regions in CRISPR/dCas9 knock-in mice. 2018. 19 (1).
8. Tanenbaum, M., et al., A protein-tagging system for signal amplification in gene expression and fluorescence imaging. 2014. 159 (3).
9. Shao, S., et al., Multiplexed sgRNA Expression Allows Versatile Single Non-repetitive DNA Labeling and Endogenous Gene Regulation. 2017. 7 (1).
10. Fu, Y., et al., CRISPR-dCas9 and sgRNA scaffolds enable dual-colour live imaging of satellite sequences and repeat-enriched individual loci. 2016. 7: p. 11707.
11. Ma, H., et al., Multiplexed labeling of genomic loci with dCas9 and engineered sgRNAs using CRISPRainbow. 2016.
12. Ma, H., et al., CRISPR-Sirius: RNA scaffolds for signal amplification in genome imaging. 2018. 15 (11).
13. Larson, D. R., et al., Real-Time Observation of Transcription Initiation and Elongation on an Endogenous Yeast Gene. 2011. 332 (6028): p. 475.
14. Dynamics of CRISPR-Cas9 genome interrogation in living cells. Science. 2015. 350 (6262): p. 823-826.
15. Ma, H., et al., CRISPR-Cas9 nuclear dynamics and target recognition in living cells. 2016: p. 529.
16. Xiaotian, et al., A CRISPR/molecular beacon hybrid system for live-cell genomic imaging. 2018. 46 (13).
17. Delehanty, J. B., et al., Delivering quantum dot-peptide bioconjugates to the cellular cytosol: escaping from the endolysosomal system. 2010. 2 (5-6): p. 265-277.
18. Santangelo, P. J., et al., Dual FRET molecular beacons for mRNA detection in living cells. 2004 (6): p. e57.
19. Piston, D. W. and G. J. J. T. i. B. S. Kremers, Fluorescent protein FRET: the good, the bad and the ugly. 2007. 32 (9): p. 407-414.
20. Muramoto, T., et al., Live imaging of nascent RNA dynamics reveals distinct types of transcriptional pulse regulation. 2012. 109 (19): p. 7350-7355.
21. Chubb, J. R., et al., Transcriptional pulsing of a developmental gene. 2006. 16 (10): p. 1018-1025.
22. Neguembor, M. V., et al., (Po) STAC (Polycistronic SunTAg modified CRISPR) enables live—cell and fixed-cell super-resolution imaging of multiple genes. 2017 (5): p. 5.
23. Hanai, R., P. R. Caron, and J. C. Wang, Human TOP3: a single-copy gene encoding DNA topoisomerase III. Proc Natl Acad Sci USA, 1996. 93 (8): p. 3653-7.
24. Ott, G., et al., The t (11;18) (q21; q21) Chromosome Translocation Is a Frequent and Specific Aberration in Low-Grade but not High-Grade Malignant Non-Hodgkin's Lymphomas of the Mucosa-associated Lymphoid Tissue (MALT-) Type. 1997. 57 (18): p. 3944-3948.
25. Wang, Y., et al., eccDNAs are apoptotic products with high innate immunostimulatory activity. Nature, 2021. 599 (7884): p. 308-314.
26. Dhungel, B. P., C. G. Bailey, and J. Rasko, Journey to the Center of the Cell: Tracing the Path of AAV Transduction. 2020.

1. A CRISPR-based target gene imaging system, comprising:
(1) a dCas9-expressing vector or a dCas9 protein;
(2) an engineered sgRNA-expressing vector, the engineered sgRNA comprising: a sgRNA backbone containing n copies of RNA aptamer, and a sgRNA sequence specific for a target gene to be detected, wherein n is an integer greater than or equal to 2; and
(3) a fusion protein-expressing vector, the fusion protein comprising: an RNA binding motif that specifically recognizes the RNA aptamer, a multimerization peptide segment and a fluorescent protein, which are operably linked.

2. The imaging system according to claim 1, wherein the engineered sgRNA-expressing vector is driven by a U6 promoter.

3. The imaging system according to claim 1, wherein the RNA aptamer and the RNA binding motif are present in a paired combination selected from the group consisting of: PP7 and PCP, MS2 and MCP or BoxB and N22.

4. The imaging system according to claim 1, wherein n is 2, 3, 4, 5, 6, 7 or 8.

5. The imaging system according to claim 1, wherein the n copies of RNA aptamer are linked in series.

6. The imaging system according to claim 1, wherein the multimerization peptide segment is foldon trimerization small peptide, GCN4 trimerization small peptide, 3HB trimerization small peptide, 6G6H hexamerization small peptide, or sDscama30 dimerization small peptide, and wherein the multimerization peptide segment is fused to the N-terminal or C-terminal of the fluorescent protein, or located at the N-terminal or C-terminal of the fusion protein.

7. The imaging system according to claim 1, wherein the fluorescent protein is green fluorescent protein (GFP), enhanced green fluorescent protein (EGFP), red fluorescent protein (RFP) or blue fluorescent protein (BFP).

8. The imaging system according to claim 1, wherein the fusion protein-expressing vector further comprises a nuclear localization sequence (NLS).

9. The imaging system of claim 1, wherein the dCas9-expressing vector is transfected into a cell line.

10. A CRISPR-based living cell target gene imaging method, the method comprising:
(i) constructing the CRISPR-based imaging system according claim 1;
(ii) transfecting a cell to be detected with each of the components in the imaging system; and
(iii) observing aggregation spots formed by the imaging system using a confocal microscope.

11. The method according to claim 10, wherein the method is used for labeling and imaging a single-copy or multi-copy gene in a living cell.

12. The method according to claim 11, wherein the gene is a chromosomal DNA or extra-chromosomal DNA.

13. The method according to claim 11, wherein the gene is an extrachromatin circular DNA element (eccDNA).

14. A kit for CRISPR-based target gene labeling and imaging, the kit comprising the dCas9-expressing vector or dCas9 protein, the engineered sgRNA-expressing vector and the fusion protein-expressing vector of the CRISPR-based imaging system according to claim 1, wherein the dCas9-expressing vector or dCas9 protein, the engineered sgRNA-expressing vector and the fusion protein-expressing vector are each stored in a separate container.

15. The imaging system according to claim 2, wherein the U6 promoter is a mouse U6 promoter (mU6) or a human U6 promoter (hU6).

16. The imaging system according to claim 5, wherein the n copies of RNA aptamer are linked in series through a linker.

17. The imaging system according to claim 6, wherein the multimerization peptide segment is located at the N-terminal of the fusion protein.
